(19) United States
(12) Patent Application Publication
(10) Pub. No.: US 2003/0162692 A1
(43) Pub. Date: Aug. 28, 2003

(54) Follicle stimulating hormone stimulated genes and uses thereof

(76) Inventor: Grace Wong, Brookline, MA (US)

Correspondence Address: Dike, Bronstein, Roberts & Cushman, Intellectual Property Patent Practice, EDWARDS & ANGELL, LLP, 130 Water Street, Boston, MA, 02109, US

(21) Appl. No.: 09/854,434

(22) Filed: May 11, 2001

Related U.S. Application Data
(60) Provisional application No. 60/203,805, filed on May 12, 2000.

(51) Int. Cl. C12Q 1/00; C12Q 1/68; A61K 38/24

(52) U.S. Cl. 514/2; 435/4; 435/6

(57) ABSTRACT

The present invention is directed to the identification genes that are expressed at a higher level in certain FSH or FSH Mimetic treated cells than in otherwise identical untreated cells. Genes that are expressed at a higher level in FSH or FSH Mimetic treated cells than untreated cells (“FSH or FSH Mimetic stimulated genes”) are of interest, in part, because FSH or FSH Mimetics can or could influence a wide range of cellular processes and responses in reproduction, including steroidogenesis and gamatogenesis. The identified FSH or FSH Mimetic stimulated genes and the proteins they encode can be used: 1) as therapeutic agents which modulate a cellular process or response that is influenced by FSH or FSH Mimetic; 2) as targets for use in high throughput screening and the development of therapeutic agents which modulate a cellular process or response that is influenced by FSH or FSH Mimetic; and 3) as markers which can be used to detect and monitor a cellular process or response that is influenced by FSH or FSH Mimetic.

This application claims the benefit of U.S. Provisional Application No. 60/203,805, filed May 12, 2000.

### BACKGROUND OF THE INVENTION

Follicle Stimulating Hormone (FSH) is a pituitary gonadotropin and is critical for the regulation of gonadal function and reproduction in humans. FSH is released in a well-orchestrated manner, under the control of numerous factors including hypothalmic gonadotropin-releasing hormone, and binds to specific high-affinity receptors in the ovary and testis to regulate gametogenesis and sex steroid synthesis and secretion.

FSH acts on the granulosa cells of the ovary and the Sertoli cells of the testis to promote germ cell development in these gonadal tissues. Accordingly, there is considerable interest in the identification of agents which can modulate cellular processes or responses influenced by FSH and markers which can be used to monitor cellular processes or responses influenced by FSH.

### SUMMARY OF THE INVENTION

The present invention is directed to the identification genes that are expressed at a higher level in certain FSH or FSH Mimetic treated cells than in otherwise identical untreated cells. Genes that are expressed at a higher level in FSH or FSH Mimetic treated cells than untreated cells (“FSH or FSH Mimetic stimulated genes”) are of interest, in part, because FSH or FSH Mimetics can or could influence a wide range of cellular processes and responses in reproduction, including steroidogenesis and gamatogenesis. The identified FSH or FSH Mimetic stimulated genes and the proteins they encode can be used: 1) as therapeutic agents which modulate a cellular process or response that is influenced by FSH or FSH Mimetic; 2) as targets for use in high throughput screening and the development of therapeutic agents which modulate a cellular process or response that is influenced by FSH or FSH Mimetic; and 3) as markers which can be used to detect and monitor a cellular process or response that is influenced by FSH or FSH Mimetic.

The FSH or FSH MIMETIC stimulated genes of the invention were identified using a nucleic acid microarray available from Incyte, Inc. and was used to determine which of approximately 8000 pre-selected nucleic acid sequences (genes) are more highly expressed in. FSH or FSH Mimetic treated Y1 cells than control cells.

Thus, the invention features a number of “FSH or FSH MIMETIC stimulated genes.” These are genes which are expressed at a relatively high level in FSH or FSH Mimetic treated Y1 cells and which are not expressed (or are expressed at a relatively low level) in otherwise identical untreated cells. These genes are listed in Tables 1-3.

In one embodiment, the invention provides genes and gene products which can be used to modulate a cellular response or process which is influenced by FSH or FSH Mimetic.

The present invention further provides genes and gene products which can be used to screen for or design agents which can be used to modulate a cellular response or process which is influenced by FSH or FSH Mimetic. Thus, the genes of the present invention (Tables 1-3) can be used as in the development of treatments (either single agent or multiple agent) for treatment of reproductive disorders. For example, if increased expression of a selected FSH or FSH Mimetic stimulated gene triggers an unwanted response, the gene or the protein encoded by the gene can be used to screen for therapeutic agents which increase or decrease expression or activity of the protein encoded by the selected gene. For example, the expression of the selected FSH or FSH Mimetic stimulated gene by an FSH or FSH Mimetic treated cell can be measured in the presence and absence of a various test agents (compounds), permitting the identification of those agents which increase or decrease expression of the selected gene.

The invention also provides markers which can be used to detect or monitor a cellular response or process that is influenced by FSH or FSH Mimetic. Thus, the markers can be used to diagnose disorders associated with an FSH or FSH Mimetic influenced cellular response or process. The markers can also be used to determine whether a selected patient suffering from a disorder associated with an FSH or FSH Mimetic influenced cellular response or process is likely to benefit from a therapy which alters the activity or expression of an FSH or FSH Mimetic stimulated gene. For example, if a given disorder is caused by increased expression of a particular FSH or FSH Mimetic stimulated gene, it may be possible to treat the disorder in patients having increased expression of the FSH or FSH Mimetic stimulated gene by decreasing expression of the FSH or FSH Mimetic stimulated gene.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described herein. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the detailed description and from the claims. Although materials and methods similar or equivalent to those described herein can be used in the practice or testing of the invention, the preferred materials and methods are described below.

### DETAILED DESCRIPTION OF THE INVENTION

The present invention is based, in part, on the identification of genes whose expression in Y1 cells are increased by treatment with FSH OR FSH Mimetic (“FSH OR FSH Mimetic stimulated genes”). The FSH or FSH Mimetic stimulated genes of the present invention as summarized in Tables I-3. Each Table is based on the Incyte, Inc. mouse cDNA array.

The increased expression of a given FSH or FSH Mimetic stimulated gene can be directly involved in or responsible for an FSH or FSH Mimetic influenced cellular response or process such that modulation of the expression or activity of the gene product will modulate the FSH or FSH Mimetic influenced cellular process or response. Such genes are generally referred to as target genes. Such genes and the products they encode can be used to modulate an FSH or FSH Mimetic influenced cellular response or process. They may also be used to develop therapeutic agents which decrease or increase the expression or activity of the protein encoded by the selected gene. Thus, the expression of the selected gene in an FSH OR FSH Mimetic treated cell can be measured in the presence and absence of a various test agents (compounds), permitting the identification of those agents which modulate expression of the selected gene. Similarly, the activity of the product of the selected gene in an FSH or FSH Mimetic treated cell can be measured in the presence and absence of a various test agents (compounds), permitting the identification of those agents which modulate the activity of the product of the selected gene The increased expression of a given FSH or FSH Mimetic stimulated gene can be associated with or correlated with a given FSH or FSH Mimetic influenced cellular response or process, but not be directly involved in the FSH or FSH Mimetic influence process or response. Modulation of the expression or activity of the protein encoded by such an FSH or FSH Mimetic stimulated gene will generally not modulate the FSH or FSH Mimetic influenced cellular process or response. Such genes and their products useful as markers which can be used to detect or monitor a cellular process or response that is influenced by FSH or FSH Mimetic. Of course, target genes and their products are similarly useful as markers which can be used to detect or monitor a cellular process or response that is influenced by FSH or FSH Mimetic.

Accordingly, the present invention provides methods for modulating an FSH or FSH Mimetic influenced cellular process or response in a patient by administering an FSH or FSH Mimetic stimulated gene (Tables 1, 2, 3) or the product thereof.

The present invention also provides a method for identifying an agent which modulates an FSH or FSH Mimetic influenced cellular process or response, the method comprising:

a) exposing a sample of cells to FSH or a FSH Mimetic;

b) determining the level of expression in the sample of cells of one or more FSH or FSH Mimetic stimulated genes (Table 1, 2, or 3) in the presence and absence of a selected agent; and

c) identifying that the agent modulates an FSH or FSH Mimetic influenced cellular process or response when the expession of the one or more FSH or FSH Mimetic stimulated genes in the cell sample in the presence of the agent differs from the expression of the one or more FSH or FSH Mimetic stimualted genes in the absence of the agent

The invention also provides a method for identifying an agent which modulates an FSH or FSH Mimetic influenced cellular process or response, the method comprising:

a) exposing a sample of cells to FSH or FSH Mimetic;

b) determining the activity in the sample of cells of the product of one or more FSH or FSH Mimetic stimulated genes (Table 1, 2, or 3) in the presence and absence of a selected agent; and identifying that the agent modulates an FSH or FSH Mimetic influenced cellular process or response when the activity of the product of the one or more FSH or FSH Mimetic stimulated genes in the cell sample in the presence of the agent differs from the activity of the product of the one or more FSH or FSH Mimetic stimulated genes in the absence of the agent.

Agents which modulate an FSH or FSH Mimetic influenced cellular process can also be identified using method which entail assessing the effect of the agent on expression or activity of FSH or FSH Mimetic stimulated genes or gene products in the absence of FSH or FSH Mimetic.

Thus, invention provides a method for identifying an agent which modulates an FSH or FSH Mimetic influenced cellular process or response, the method comprising:

a) providing a sample of cells;

b) determining the level of expression in the sample of cells of one or more FSH or FSH Mimetic stimulated genes (Table 1, 2, or 3) in the presence and absence of a selected agent; and

c) identifying that the agent modulates an FSH or FSH Mimetic influenced cellular process or response when the expression of the one or more FSH or FSH Mimetic stimulated genes in the cell sample in the presence of the agent differs from the expression of the one or more FSH or FSH Mimetic stimulated genes in the absence of the agent.

The invention also provides a method for identifying an agent which modulates an FSH or FSH Mimetic influenced cellular process or response, the method comprising:

a) providing a sample of cells;

b) determining the activity in the sample of cells of the product of one or more FSH or FSH Mimetic stimulated genes (Table 1) in the presence and absence of a selected agent; and

c) identifying that the agent modulates an FSH or FSH Mimetic influenced cellular process or response when the activity of the product of the one or more FSH or FSH Mimetic stimulated genes in the cell sample in the presence of the agent differs from the activity of the product of the one or more FSH or FSH Mimetic stimulated genes in the absence of the agent.

In all of the above-described methods for identifying an agent which modulates an FSH or FSH Mimetic influenced cellular process or response, the preferred FSH or FSH Mimetic stimulated genes are those which are target genes.

The invention also provides a method for detecting or monitoring a cellular process or response that is influenced by FSH or FSH Mimetic, the method comprising:

a) obtaining a sample of cells from a patient;

b) determining the level of expression in the sample of cells of one or more FSH or FSH Mimetic stimulated genes (Tables 1, 2, 3); and

c) identifying that the cells in the sample of cells obtained from the patient are undergoing a cellular process or response that is influenced by FSH or FSH Mimetic when the level of expression of the one or more FSH or FSH Mimetic stimulated genes in the cell sample is increased relative to the activity of the one or more FSH or FSH Mimetic stimulated genes in a control the sample.

The invention also provides a method for detecting or monitoring a cellular process or response that is influenced by FSH or FSH Mimetic, the method comprising:

a) obtaining a sample of cells from a patient;

b) determining the level of activity in the sample of cells of the product of one or more FSH or FSH Mimetic stimulated genes (Table 1, 2, or 3); and

c) identifying that the cells in the sample of cells obtained from the patient are undergoing a cellular process or response that is influenced by FSH or FSH Mimetic when the level of activity of the product of the one or more FSH or FSH Mimetic stimulated genes in the cell sample is increased relative to the activity of the product of the one or more FSH or FSH Mimetic stimulated genes in the control sample.

### Measurement of Expression in Diagnostic or Screening Methods

As used herein, the level or amount of expression of a gene refers to the absolute level of expression of a mRNA encoded by the gene or the absolute level of expression of the protein encoded by the gene.

Often, it is preferable to determine the expression of two or more of the identified genes, more preferably, three or more of the identified genes. Thus, it is preferable to assess the expression of a panel of genes.

As an alternative to making determinations based on the absolute expression level of selected gene(s), e.g, one or more FSH or FSH Mimetic stimulated genes selected from the genes of Tables 1, 2, 3 determinations may be based on the normalized expression levels. Expression levels are normalized by correcting the absolute expression level of an FSH or FSH Mimetic stimulated gene or a by comparing its expression to the expression of a gene that is not an FSH or FSH Mimetic stimulated gene, e.g., a housekeeping genes that is constitutively expressed. Suitable genes for normalization include housekeeping genes such as the actin gene. This normalization allows one to compare the expression level in one sample, e.g., a patient sample, to another sample, e.g., a patient sample collected at an earlier time, or between samples from different sources.

Alternatively, the expression level can be provided as a relative expression level. To determine a relative expression level of a gene, the level of expression of the gene is determined for 10 or more samples, preferably 50 or more samples, prior to the determination of the expression level for the sample in question. The mean expression level of each of the genes assayed in the larger number of samples is determined and this is used as a baseline expression level for the gene(s) in question. The expression level of the gene determined for the test sample (absolute level of expression) is then divided by the mean expression value obtained for that gene.

Preferably, the samples used will be from similar tissues. The choice of the cell source is dependent on the use of the relative expression level data. For example, in order to determine whether a particular tissue will be relatively affected, using tissues of similar types for obtaining a mean expression score is preferred.

Using expression found in normal cells or cells which are not exposed to FSH or FSH Mimetic as a mean expression score aids in validating whether the gene assayed is specific for an FSH or FSH Mimetic influenced cellular process or response. Such a later use is particularly important in identifying whether a given FSH or FSH Mimetic stimulated gene can serve as a target gene. In addition, as more data is accumulated, the mean expression value can be revised, providing improved relative expression values based on accumulated data.

The expression level can be measured in a number of ways, including, but not limited to: measuring the mRNA encoded by the selected genes; measuring the amount of protein encoded by the selected genes; or measuring the activity of the protein encoded by the selected genes.

The mRNA level can be determine in in situ and in in vitro formats using methods known in the art. Many of such methods use isolated RNA. For in vitro methods, any RNA isolation technique that does not select against the isolation of mRNA can be utilized for the purification of RNA from gonadal cells (see, e.g., Ausubel et al., eds., 1987-1997, Current Protocols in Molecular Biology,

John Wiley & Sons, Inc. New York). Additionally, large numbers of tissue samples can readily be processed using techniques well known to those of skill in the art, such as, for example, the single-step RNA isolation process of Chomczynski (I 989, U.S. Pat. No. 4,843,155).

The isolated mRNA can be used in hybridization or amplification assays that include, but are not limited to, Southern or Northern analyses, polymerase chain reaction analyses and probe arrays. One preferred diagnostic methods for the detection of mRNA levels involves contacting the isolated mRNA with a nucleic acid molecule (probe) that can hybridize to the mRNA encoded by the gene being detected. In one format, the mRNA is immobilized on a solid surface and contacted with the probes, for example by running the isolated mRNA on an agarose gel and transferring the mRNA from the gel to a membrane, such a nitrocellulose. In an alternative format, the probes are immobilized on a solid surface and the mRNA is contacted with the probes, for example in a gene microarray of the type available from Incyte, Inc. A skilled artisan can readily adapt known mRNA detection methods for use in detecting the level of mRNA encoded by one or more of the identified FSH OR FSH Mimetic stimulated genes.

An alternative method for determining the level of mRNA in a sample that is encoded by one of the genes of the present invention involves the process of nucleic acid amplification, e.g., by rtPCR (the experimental embodiment set forth in Mullis, 1987, from about 50 to 200 nucleotides in length). Under appropriate conditions with appropriate reagents, amplification primers result in the production of nucleic acid molecule comprising the nucleotide sequence flanked by the primers. A skilled artisan can readily determine appropriate primers (both nucleotide sequence and length) for amplifying and detecting the FSH or FSH Mimetic stimulated genes of the present invention using art known methods and the nucleotide sequence of the FSH or FSH Mimetic stimulated genes of the present invention.

A variety of methods can be used to determine the level of protein encoded by one or more of the FSH or FSH Mimetic stimulated genes of the present invention. In general, these methods involve the use of a compound that selectively binds to the protein, for example an antibody.

Proteins can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of formats can be employed to determine whether a sample contains a protein that binds to a given antibody. Examples of such formats include, but are not limited to enzyme immunoassay (EIA), radioimmunoassay (RIA), Western blot analysis and enzyme linked immunoabsorbant assay (ELISA). A skilled artisan can readily adapt know protein/antibody detection methods for use in determining whether a cells expresses a protein encoded by one or more of the genes of the present invention.

In one format, antibodies, or antibody fragments, can be used in methods such as Western blots or immunofluorescence techniques to detect the expressed proteins. In such uses, it is generally preferable to immobilize either the antibody or protein on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, proteins isolated from cells can be run on a polyacrylamide gel electrophoresis and immobilized onto a solid phase support such as nitrocellulose. The support can then be washed with suitable buffers followed by treatment with the delectably labeled antibody that selectively binds a protein encoded by an FSH or FSH Mimetic stimulated gene of the invention. The solid phase support can then be washed with the buffer a second time to remove unbound antibody. The amount of bound label on the solid support can then be detected by conventional means.

Another embodiment of the present invention includes a step of detecting whether an agent alters the expression of one or more of the FSH or FSH Mimetic stimulated genes of the present invention. Although the present FSH or FSH Mimetic stimulated genes were identified as being expressed in cells that were not being exposed to a potential therapeutic agent, treatment with an agent may, or may not, alter expression. Such alterations in the expression level of these genes can provide a further indication as to whether the cells will be responsive to treatment with the agent. In such a use, the present invention provides methods for assessing whether the cells will be responsive to an agent which modulates an FSH or FSH Mimetic influenced cellular process or response, the method comprising:

a) exposing a sample of cells obtained from a patient to a test agent;

b) determining the level of expression of one or more genes FSH or FSH Mimetic stimulated genes (Tables 1, 2, 3) in the sample of cells exposed to the agent and in a sample of cells that is not exposed to the agent; and

c) determining that the cells will be responsive to the agent when expression of the one or more of the FSH or FSH Mimetic stimulated genes in sample of cells exposed to the agent differs from the expression of the one or more FSH or FSH Mimetic stimulated genes in the sample of cells not exposed to the agent.

This embodiment of the methods of the present invention involves the step of exposing the cells to an agent. The method used for exposing the reproductive cells to the agent will be based primarily on the source and nature of the cells and the agent being tested. The contacting can be performed in vitro or in vivo, in a patient being treated/evaluated or in animal model of a reproductive disorder. For cells and cell lines and chemical compounds, exposing the cells involves contacting the cells with the compound, such as in tissue culture media. A skilled artisan can readily adapt an appropriate procedure for contacting cells with any particular agent or combination of agents.

As discussed above, the identified FSH or FSH Mimetic stimulated genes of the invention can be used as markers to monitor an FSH OR FSH Mimetic influenced cellular process or response. For example, exposure to FSH or FSH Mimetic can increase gamatogenesis. Accordingly, by monitoring the expression of FSH or FSH Mimetic stimulated genes one can assess whether a gonadal tissue has become reponsive or refractory to an ongoing treatment. When gonadal tissue is no longer responding to a treatment the expression profile of the gonadal tissue will change: the level of expression of one or more of the FSH OR FSH Mimetic stimulated genes will decrease.

In such a use, the invention provides methods for determining whether an treatment should be continued in a patient, comprising the steps of

a) obtaining a first sample of tissue from a patient undergoing therapy at a first time and obtaining a second sample of cells from the patient at a second later time;

b) determining the level of expression of one or more genes FSH or FSH Mimetic stimulated genes (Table 1, 2, 3) in the first and second samples of cells; and

c) discontinuing treatment when the expression of one or more FSH or FSH Mimetic stimulated genes is lower in the second sample of cells than in the first sample of cells.

As used here, a patient refers to any subject undergoing treatment for reproductive disorder. The preferred subject will be a human patient undergoing FSH replacement therapy.

This embodiment of the present invention relies on comparing two or more samples obtained from a patient undergoing FSH replacement therapy. In general, it is preferable to obtain a first sample from the patient prior to beginning therapy and one or more samples during treatment. In such a use, a baseline of expression prior to therapy is determined and then changes in the baseline state of expression is monitored during the course of therapy. Alternatively, two or more successive samples obtained during treatment can be used without the need of a pre-treatment baseline sample. In such a use, the first sample obtained from the subject is used as a baseline for determining whether the expression of a particular gene is increasing or decreasing.

In general, when monitoring the effectiveness of a therapeutic treatment, two or more samples from the patient are examined. Preferably, three or more successively obtained samples are used, including at least one pretreatment sample.

### Kits Containing Reagents For Conducting the Methods of the Present Invention

The present invention further provides kits comprising compartmentalized containers comprising reagents for detecting one or more, preferably two or more, of the FSH or FSH Mimetic stimulated genes of the present invention. As used herein a kit is defined as a prepackaged set of containers into which reagents are placed. The reagents included in the kit comprise probes/primers and/or antibodies for use in detecting FSH or FSH Mimetic stimulated gene expression. In addition, the kits of the present invention may preferably contain instructions which describe the use of the kit. Such kits can be conveniently used, e.g., in clinical settings, to diagnose patients exhibiting, e.g., symptoms of a disorder associated with an FSH influenced cellular process or response.

### Further Characterization of FSH or FSH Mimetic Stimulated Genes

FSH or FSH Mimetic stimulated genes of the present invention can be further characterized using techniques known to those skilled in the art to yield more information regarding potential targets for the therapeutic modulation of an FSH or FSH Mimetic influenced cellular process or response, steroidogenesis or gamatogenesis. For example, characterization of the identified genes can yield information regarding the biological function of the identified genes.

Specifically, any of the FSH or FSH Mimetic stimulated genes whose further characterization indicates that a modulation of the gene's expression or a modulation of the gene product's activity modulate an FSH OR FSH Mimetic influence cellular process or response are designated “target genes.” Target genes and target gene products can be used to identify therapeutics agents.

An FSH or FSH Mimetic stimulated gene whose further characterization indicates that it does not modulate an FSH or FSH Mimetic influenced cellular process or response, but whose expression pattern contributes to a gene expression pattern correlative of, an FSH or FSH Mimetic influenced cellular process or response cannot serve as a target gene. Such genes can be used as diagnostic markers and as markers for assessing or monitoring an FSH or FSH Mimetic influenced cellular process or response.

A variety of techniques can be utilized to further characterize the genes herein identified. First, the nucleotide sequence of the identified genes, obtained by standard techniques well known to those of skill in the art, can be used to further characterize such genes. For example, the sequence of the identified genes can reveal homologies to one or more known sequence motifs that can yield information regarding the biological function of the identified gene product.

Second, an analysis of the tissue and/or cell type distribution of the mRNA produced by the identified genes can be conducted, utilizing standard techniques well known to those of skill in the art. Such techniques can include, for example, Northern analyses, RT-coupled PCR and RNase protection techniques. Such analyses can be used to determine whether cells within a given tissue express the identified gene. Such an analysis can also provide information regarding the biological function of an identified gene.

Third, the sequences of the identified genes can be used, utilizing standard techniques, to place the genes onto genetic maps, e.g., mouse (Copeland and Jenkins 1991, Trends in Genetics 7:113-118) and human genetic maps (Cohen et al., 1993, Nature 366:698-701). Such mapping information can yield information regarding the genes' importance to human disease by, for example, identifying genes that map within a genetic region to which predisposition to reproductive disorders also maps.

Fourth, the biological function of the identified genes can be more directly assessed by utilizing relevant in vivo and in vitro systems. In vivo systems can include, but are not limited to, animal systems that naturally exhibit symptoms of a disorder of interest, e.g., an immune disorder or a proliferative disorder or ones that have been engineered to exhibit such symptoms. The role of identified gene products can be determined by transfecting cDNAs encoding these gene products into appropriate cell lines and analyzing the effect of the gene product on the cells.

In further characterizing the biological function of the identified genes, the expression of these genes can be modulated within the in vivo and/or in vitro systems, i.e., either over-expressed or under-expressed, and the subsequent effect on the system then assayed. Alternatively, the activity of the product of the identified gene can be modulated by either increasing or decreasing the level of activity in the in vivo and/or in vitro system of interest, and assessing the effect of such modulation.

The information obtained through such characterizations can suggest relevant methods for the modulation of an FSH or FSH Mimetic influenced cellular response or process, e.g., steroidogenesis or gamatogenesis. For example, treatment can include a modulation of gene expression and/or gene product activity. Characterization procedures such as those described herein can indicate where such modulation should involve an increase or a decrease in the expression or activity of the gene or gene product of interest.

### Identification of Compounds that Interact with a Target Gene Product

The following assays are designed to identify compounds that bind to target gene products, compounds that bind to other cellular proteins that interact with a target gene product, and compounds that interfere with the interaction of the target gene product with other cellular proteins. Such compounds can include, but are not limited to, other cellular proteins, natural products and small chemical molecules. Specifically, such compounds can include, but are not limited to, peptides, such as, for example, soluble peptides, including, but not limited to Ig-tailed fusion peptides, comprising extracellular portions of target gene product transmembrane receptors, and members of random peptide libraries (see, e.g., Lam et al., 1991, Nature 354:82-84; Houghton et al., 1991, Nature 354:84-86), made of D-and/or L-configuration amino acids, phosphopeptides (including, but not limited to, members of random or partially degenerate phosphopeptide libraries; see, e.g., Songyang et al., 1993, Cell 72:767-778), antibodies (including, but not limited to, polyclonal, monoclonal, humanized, anti-idiotypic, chimeric or single chain antibodies, and FAb, F(ab′)2and FAb expression library fragments, and epitope-binding fragments thereof), and small organic or inorganic molecules.

Compounds identified via assays such as those described herein can be useful, for example, in elaborating the biological function of the target gene product, and for modulating steroidogenesis or gamatogenesis. For example, for FSH or FSH Mimetic stimulated genes that are target genes, compounds that decrease the level of expression of the gene or the activity of the encoded protein, are potential therapeutic agents for reproductive disorders resulting from increased levels of FSH. Conversely, compounds that increase the level of expression of the gene or the activity of the encoded protein, are potential therapeutic agents for reproductive disorders requiring increased steroidogenesis and/or gamatogenesis.

### Screening Assays For Compounds and Cellular Proteins That Bind to a Target Gene Product

In vitro systems can be designed to identify compounds capable of binding the target gene products of the invention. Compounds thus identified can be used to modulate the activity of target gene products in a therapeutic protocol, to elaborate the biological function of the target gene product, or to identify compounds that disrupt normal target gene interactions. The preferred targets genes/products used in this embodiment are the FSH or FSH Mimetic stimulated genes of the present invention.

The principle of the assays used to identify compounds that bind to the target gene product involves preparing a reaction mixture of the target gene protein and the test compound under conditions and for a time sufficient to allow the two components to interact and bind, thus forming a complex that can be removed and/or detected in the reaction mixture. These assays can be conducted in a variety of ways. For example, one method to conduct such an assay would involve anchoring target gene product or the test substance onto a solid phase and detecting target gene product/test compound complexes anchored on the solid phase at the end of the reaction. In one embodiment of such a method, the target gene product can be anchored onto a solid surface, and the test compound, that is not anchored, can be labeled, either directly or indirectly.

In practice, microliter plates can conveniently be utilized as the solid phase. The anchored component can be immobilized by non-covalent or covalent attachments. Non-covalent attachment can be accomplished by simply coating the solid surface with a solution of the protein and drying. Alternatively, an immobilized antibody, preferably a monoclonal antibody, specific for the protein to be immobilized can be used to anchor the protein to the solid surface. The surfaces can be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously immobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the immobilized component (the antibody, in turn, can be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for target gene or the test compound to anchor any complexes formed in solution, and a labeled antibody specific for the other component of the possible complex to detect anchored complexes.

Any method suitable for detecting protein-protein interactions can be employed for identifying novel target product-cellular or extracellular protein interactions. In such a case, the target gene serves as the known “bait” gene.

### Assays For Compounds That Interfere with the Binding of a Target Gene Product to a Second Cellular Protein

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules.

The basic principle of the assay systems used to identify compounds that interfere with the interaction between the target gene product and its cellular or extracellular binding partner or partners involves preparing a reaction mixture containing the target gene product, and the binding partner under conditions and for a time sufficient to allow the two products to interact and bind, thus forming a complex. In order to test an agent for inhibitory activity, the reaction mixture is prepared in the presence and absence of the test compound. The test compound can be initially included in the reaction mixture, or can be added at a time subsequent to the addition of target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

The assay for compounds that interfere with the interaction of the target gene products and binding partners can be conducted in a heterogeneous or homogeneous format. Heterogeneous assays involve anchoring either the target gene product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance; i.e., by adding the test substance to the reaction mixture prior to or simultaneously with the target gene product and interactive cellular or extracellular binding partner. Alternatively, test compounds that disrupt preformed complexes, e.g., compounds with higher binding constants that displace one of the components from the complex, can be tested by adding the test compound to the reaction mixture after complexes have been formed. The various formats are described briefly below.

In a heterogeneous assay system, either the target gene product or the Interactive cellular or extracellular binding partner, is anchored onto a solid surface, while the non-anchored species is labeled, either directly or indirectly. In practice, microtitre plates are conveniently utilized. The anchored species can be immobilized by noncovalent or covalent attachments. Non-covalent attachment can be accomplished simply by coating the solid surface with a solution of the target gene product or binding partner and drying. Alternatively, an immobilized antibody specific for the species to be anchored can be used to anchor the species to the solid surface. The surfaces can be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the nonimmobilized species is pre-labeled, the detection of label immobilized on the surface indicates that complexes were formed. Where the non-immobilized species is not prelabeled, an indirect label can be used to detect complexes anchored on the surface; e.g., using a labeled antibody specific for the initially non-immobilized species (the antibody, in turn, can be directly labeled or indirectly labeled with a labeled anti-Ig antibody). Depending upon the order of addition of reaction components, test compounds that inhibit complex formation or that disrupt preformed complexes can be detected. Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. In this approach, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-cellular or extracellular binding partner interaction can be identified.

### Assays Based on Target Gene Product Activity

The present invention further provides methods for identifying new therapeutic agents, or combinations of therapeutic agents, that modulate the activity or expression of one or more the gene products encoded the FSH or FSH Mimetic stimulated genes of the invention. Specifically, the activity of the proteins encoded by the genes of the present invention can be used as a basis for identifying agents which can be used to modulate an FSH or FSH Mimetic influenced cellular process or response, e.g. steroidogenesis and/or gamatogenesis. For example, by blocking the activity of one or more of the proteins encoded by FSH or FSH Mimetic stimulated genes of the invention, reproductive cells will become sensitive to treatment with an agent that the unmodified reproductive cells were resistant to.

The choice of assay format will be based primarily on the nature and type of protein being assayed. A skilled artisan can readily adapt protein activity assays for use in the present invention with the genes identified herein.

### Modulation of an FSH or FSH Mimetic Influenced Cellular Process or Response by Modulation of FSH or FSH Mimetic Stimulated Genes or Gene Products

FSH or FSH Mimetic influenced cellular processes and responses, e.g., steroidogenesis and/or gamatogenesis, can be modulated by modulating the expression of a target gene or the activity of a target gene product. The modulation can be of a positive or negative nature, depending on the specific situation involved.

“Negative modulation,” refers to a reduction in the level and/or activity of target gene product relative to the level and/or activity of the target gene product in the absence of the modulatory treatment.

“Positive modulation,” refers to an increase in the level and/or activity of target gene product relative to the level and/or activity of target gene product in the absence of modulatory treatment.

It is possible that a disorder associated with an FSH or FSH Mimetic influenced cellular process or response, can be caused, at least in part, by an abnormal level of a target gene product, or by the presence of a gene product exhibiting abnormal activity. As such, the reduction in the level and/or activity of such gene products would bring about the amelioration of the disorder. Alternatively, it is possible that such disorders can be brought about, at least in part, by the absence or reduction of the level of target gene expression, or a reduction in the level of a gene product's activity. As such, an increase in the level of gene expression and/or the activity of such gene products would bring about the amelioration of the disorder. As discussed, above, successful treatment of various disorders can be brought about by techniques that serve to inhibit the expression or activity of target gene products (i e., the product of FSH or FSH Mimetic stimulated genes that are target genes).

For example, compounds, e.g., an agent identified using an assays described above, that proves to exhibit negative modulatory activity, can be used in accordance with the invention to prevent and/or ameliorate symptoms of a disorder associated with an FSH or FSH Mimetic influences cellular process or response, e.g., steroidogenesis and/or gamatogenesis. Such molecules can include, but are not limited to peptides, phosphopeptides, small organic or inorganic molecules, or antibodies (including, for example, polyclonal, monoclonal, humanized, anti-idlotypic, chimeric or single chain antibodies, and FAb, F(ab′)₂ and FAb expression library fragments, and epitope-binding fragments thereof.

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity,

Among the compounds that can be used to reduce or inhibit either wild type, or if appropriate, mutant target gene activity are antisense, ribozyme, and triple helix molecules. Techniques for the production and use of such molecules are well known to those of skill in the art.

Anti-sense RNA and DNA molecules act to directly block the translation of mRNA by hybridizing to targeted mRNA and preventing protein translation. With respect to antisense DNA, oligodeoxyribonucleotides derived from the translation initiation site, e.g., between the −10 and +10 regions of the target gene nucleotide sequence of interest, are preferred.

Ribozymes are enzymatic RNA molecules capable of catalyzing the specific cleavage of RNA. (For a review, see, for example, Rossi, 1994, Current Biology 4:469-471.) The mechanism of ribozyme action involves sequence specific hybridization of the ribozyme molecule to complementary target RNA, followed by a endonucleolytic cleavage. The composition of ribozyme molecules must include one or more sequences complementary to the target gene mRNA and must include the well-known catalytic sequence responsible for mRNA cleavage. For this sequence, see U.S. Pat. No. 5,093,246, that is incorporated by reference herein in its entirety. As such within the scope of the invention are engineered hammerhead motif ribozyme molecules that specifically and efficiently catalyze endonucleolytic cleavage of RNA sequences encoding target gene proteins.

Specific ribozyme cleavage sites within any potential RNA target are initially identified by scanning the molecule of interest for ribozyme cleavage sites that include the following sequences, GUA, GUU, and GUC. Once identified, short RNA sequences of between 15 and 20 ribonucleotides corresponding to the region of the target gene containing the cleavage site can be evaluated for predicted structural features, such as secondary structure, that can render the oligonucleotide sequence unsuitable. The suitability of candidate sequences can also be evaluated by testing their accessibility to hybridization with complementary oligonucleotides, using ribonuclease protection assays.

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, that generally require sizeable stretches of either purines or pyrimidines to be present on one strand of a duplex. Nucleotide sequences can be pyrimidine-based, that will result in TAT and CGC′ triplets across the three associated strands of the resulting triple helix. The pyrimidine-rich molecules provide base complementarily to a purine-rich region of a single strand of the duplex in a parallel orientation to that strand. In addition, nucleic acid molecules can be chosen that are purine-rich, for example, contain a stretch of G residues. These molecules will form a triple helix with a DNA duplex that is rich in GC pairs, in that the majority of the purine residues are located on a single strand of the targeted duplex, resulting in GGC triplets across the three strands in the triplex. Alternatively, the potential sequences that can be targeted for triple helix formation can be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or 25 pyrimidines to be present on one strand of a duplex.

In instances wherein the antisense, ribozyme, and/or triple helix molecules described herein are utilized to reduce or inhibit mutant gene expression, it is possible that the technique utilized can also efficiently reduce or inhibit the transcription (triple helix) and/or translation (antisense, ribozyme) of mRNA produced by normal target gene alleles such that the possibility can arise wherein the concentration of normal target gene product present can be lower than is necessary for a normal phenotype. In such cases, to ensure that substantially normal levels of target gene activity are maintained, nucleic acid molecules that encode and express target gene polypeptides exhibiting normal target gene activity can be introduced into cells via gene therapy method. Alternatively, in instances in that the target gene encodes an extracellular protein, it can be preferable to co-administer normal target gene protein into the cell or tissue in order to maintain the requisite level of cellular or tissue target gene activity.

Anti-sense RNA and DNA, ribozyme and triple helix molecules of the invention can be prepared by any method known in the art for the synthesis of DNA and RNA molecules. These include techniques for chemically synthesizing oligodeoxyribonucleotides and oligoribonucleotides well known in the art such as, for example, solid phase phosphoramidite chemical synthesis. Alternatively, RNA molecules can be generated by in vitro and in vivo transcription of DNA sequences encoding the antisense RNA molecule. Such DNA sequences can be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications to the DNA molecules can be introduced as a means of increasing intracellular stability and half-life. Possible modifications include but are not limited to the addition of flanking sequences of ribo- or deoxynucleotides to the 5′ and/or 3′ ends of the molecule or the use of phosphorothioate or 2′ O-methyl rather than phosphodiesterase linkages within the oligodeoxyribonucleotide backbone.

Antibodies can be generated that are both specific for target gene product and that reduce target gene product activity. Such antibodies may, therefore, by administered in instances whereby negative modulatory techniques are appropriate. Antibodies can be generated using standard techniques against the proteins themselves or against peptides corresponding to portions of the proteins. The antibodies include but are not limited to polyclonal, monoclonal, Fab fragments, single chain antibodies, chimeric antibodies, and the like.

In instances where the target gene protein to that the antibody is directed is intracellular and whole antibodies are used, internalizing antibodies can be preferred. However, lipofectin or liposomes can be used to deliver the antibody or a fragment of the Fab region that binds to the target gene epitope into cells. Where fragments of the antibody are used, the smallest inhibitory fragment that binds to the target protein's antibinding domain is preferred. For example, peptides having an amino acid sequence corresponding to the domain of the variable region of the antibody that binds to the target gene protein can be used. Such peptides can be synthesized chemically or produced via recombinant DNA technology using methods well known in the art (e.g., see Creighton, 1983, supra; and Sambrook et al., 1989, supra). Alternatively, single chain neutralizing antibodies that bind to intracellular target gene product epitopes can also be administered. Such single chain antibodies can be administered, for example, by expressing nucleotide sequences encoding single-chain antibodies within the target cell population by utilizing, for example, techniques such as those described in Marasco et al. (1993, Proc. Natl. Acad. Sci. USA 90:7889-7893).

### Therapeutic Treatment

The identified compounds that modulate target gene expression, synthesis and/or activity can be administered to a patient at therapeutically effective doses to prevent, treat or ameliorate a disorder associated with an FSH OR FSH Mimetic influenced cellular process or response. A therapeutically effective dose refers to that amount of the compound sufficient to result in amelioration of symptoms of the disorder.

### Effective Dose

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50 Compounds that exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

### Formulations and Use

Pharmaceutical compositions for use in accordance with the present invention can be formulated in conventional manner using one or more physiologically acceptable carriers or excipients.

Thus, the compounds and their physiologically acceptable salts and solvates can be formulated for administration by inhalation or insufflation (either through the mouth or the nose) or oral, buccal, parenteral or rectal administration.

For oral administration, the pharmaceutical compositions can take the form of, for example, Tablets or capsules prepared by conventional means with pharmaceutically acceptable excipients such as binding agents (e.g., pregelatinised maize starch, polyvinylpyrrolidone-or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The Tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the compositions can take the form of Tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example as an emulstion in an acceptable oil) or ion excange resins, or as sparingly soluble derivatives, for example, as a sparingly soluble salt.

The compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

### EXAMPLE

### Identification of FSH OR FSH Mimetic Stimulated Genes

A mouse cDNA array from Incyte, Inc. was used to analyze the expression profile of Y-1 (ATCC Accession No. CCL 79) cells. This transcriptional profile analysis led to the identification of the FSH or FSH Mimetic stimulated genes of the invention.

Y1 cells were incubated with Ham's F-10 Nutrient Mixture Media supplemented with glutamine (1%), G418 geneyticin (80 ug/ml), penicillin-streptomycin (1%), fetal horse serum (15%), and fetal bovine serum (2.5%) and grown to confluency. Confluent cells were then exposed to FSH at 10-9 M (See Table 1) or FSH Mimetic 024 at 1 uM (see Table 3), Forskolin at 1 uM (See Table 4) and all three agents (i.e., FSH at 10⁻⁹ M, FSH Mimetic 024 at 1 uM and Forskolin at 1 uM—See Table 2) for 16 hours. Total RNA was isolated from control or treated cells with ULTRASPEC™ RNA from Biotech Laboratories, Inc. All media and supplements were obtained from Gibco BRL Life Technologies.

The GEM (Gene Expression Microarray) technology uses the following steps to discover differences in gene expression between two messenger RNA (mRNA) samples. Small samples of cDNA were deposited on a glass surface and bonded to the glass. Subsequently, large portions from one half of the DNA's double strands were removed in order to activate the individual elements of the array, preparing them to react and bind to their uniquely matched DNA counterparts in the cells being tested.

Two mRNA samples were prepared and color labeled, since the GEM technology uses a color coding technique to discover the differences in gene expression between two mRNA samples. Messenger RNA was extracted from the normal or unaffected sample, and a fluorescent labeled cDNA probe was generated. The probe represents all of the genes expressed in the reference sample. Next, the mRNA was extracted from another sample. Typically, were are the affected cells (e.g., Y-1 cells): exposed to FSH or FSH Mimetic or removed at a different time. The fluorescent labeling step was repeated to generate a second cDNA probe using a different color fluorescent molecule.

The two fluorescent probe samples were simultaneously applied to a single microarray, where they competitively react with the arrayed cDNA molecules. Following incubation, the microarray was rinsed, washing off those probe molecules that did not find their cDNA counterpart. Each element of the GEM microarray was scanned for the first fluorescent color. The intensity of the fluorescence at each array element is proportional to the expression level of that gene in the sample. The scanning operation was repeated for the second fluorescent label. The ratio of the two fluorescent intensities provided a quantitative measurement of the relative gene expression level in the two cell samples. For example, if a microarray element shows no color, it indicated that the gene in that element was not expressed in either cell sample. If an element showed a single color, it indicated that a labeled gene was expressed only in that cell sample. The appearance of both colors indicated that the gene was expressed in both cell samples.

The genes identified on the GEM array that are more highly expressed in Y-1 treated cells than control cells are listed in Tables 1, 2, 3 and 4 in order of decreasing differential expression. Each entry includes the I.M.A.G.E. Database Accession Number for the sequence. I.M.A.G.E. clones can be obtained from, e.g., the American Type Culture Collection (Manassas, V.).

TABLE 1
FSH regulats 189 genes in Y1 cells

| Fold | Con | FSH | GeneName |
|---|---|---|---|
| −7.9 | 73 | 576 | IMAGE EST {IMAGE:598824} |
| −6.9 | 665 | 4571 | MYELOID DIFFERENTIATION PRIMARY RESPONSE PROTEIN MYD116 {IMAGE:475803} |
| −5 | 973 | 4856 | Extracellular matrix protein 1 {IMAGE:874833} |
| −4.6 | 1297 | 5965 | Extracellular matrix protein 1 {IMAGE:678765} |
| −4.6 | 962 | 4378 | ESTs, Weakly similar to cDNA EST EMBL:D75506 comes from this gene [C. elegans] {IMAGE:334182} |
| −3.8 | 426 | 1599 | NAD-DEPENDENT METHYLENETETRAHYDROFOLATE DEHYDROGENASE {IMAGE:406031} |
| −3.8 | 103 | 395 | ESTs, Highly similar to CYSTATHIONINE GAMMA-LYASE [Homo sapiens ] {IMAGE:679235} |
| −3.7 | 206 | 764 | Musmusculus secreted carbonic anhydrase isozyme VI precursor, mRNA, complete cds {IMAGE:32994 |
| −3.3 | 403 | 1335 | ESTs {IMAGE:482641} |
| −3.3 | 308 | 1003 | ESTs, Highly simliar to SERINE HYDROXYMETHYLTRANSFERASE, MITOCHONDRIAL [Oryctolagus cu [symbol] |
| −3.2 | 2198 | 6995 | Antigen identified by monoclonal antibodies 4F2 {IMAGE:478301} |
| −3.2 | 311 | 1003 | ESTs {IMAGE:426033} |
| −3.1 | 414 | 1275 | IMAGE EST {IMAGE:656089} |
| −3.1 | 144 | 444 | IMAGE EST {IMAGE:818790} |
| −3.1 | 93 | 287 | IMAGE EST {IMAGE:574227} |
| −3 | 76 | 227 | ESTs {IMAGE:749313} |
| −2.9 | 825 | 2419 | Mouse mRNA for dbpA murine homologue, complete cds {IMAGE:481949} |
| −2.9 | 405 | 1181 | IMAGE EST {IMAGE:315676} |
| −2.9 | 341 | 982 | IMAGE EST {IMAGE:467785} |
| −2.9 | 159 | 468 | ESTs {IMAGE:876063} |
| −2.8 | 545 | 1539 | Mus musculus A10 mRNA, partial cds {IMAGE:385441} |
| −2.8 | 319 | 895 | IMAGE EST {IMAGE:367765} |
| −2.7 | 327 | 874 | ESTs, Highly similar to CYSTATHIONINE GAMMA-LYASE [Homo sapiens ] {IMAGE:525119} |
| −2.6 | 801 | 2085 | Glutamate oxaloacetate transaminase 1, soluble {IMAGE:481381} |
| −2.6 | 643 | 1660 | M. musculus mRNA for mTGIF protein {IMAGE:474339} |
| −2.5 | 1416 | 3485 | Heat shock protein, 74 kDa {IMAGE:444027} |
| −2.5 | 776 | 1924 | Myelocytomatosis oncogene {IMAGE:441346} |
| −2.5 | 754 | 1856 | Mouse chromatin nonhistone high mobility group protein (HGM-I(Y), complete cds {IMAGE:920268} |
| −2.5 | 590 | 1478 | Glycine transporter 1 {IMAGE:420070} |
| −2.5 | 465 | 1142 | IMAGE EST {IMAGE:681424} |
| −2.5 | 441 | 1084 | Hormone receptor {IMAGE:439773} |
| −2.5 | 313 | 770 | ESTs {IMAGE:404057} |
| −2.5 | 305 | 769 | ESTs, Weakly similar to Tid56 protein [D.melanogaster] {IMAGE:478167} |
| −2.5 | 182 | 450 | Heme oxygenase (decycling) 1 {IMAGE:677499} |
| −2.4 | 452 | 1073 | ESTs, Highly similar to PUTATIVE ASPARAGINYL-TRNA SYNTHETASE DED81 [Saccharomyces cerev |
| −2.4 | 351 | 836 | IMAGE EST {IMAGE:479247} |
| −2.4 | 310 | 752 | IMAGE EST {IMAGE:640085} |
| −2.3 | 621 | 1402 | IMAGE EST {IMAGE:466678} |
| −2.3 | 514 | 1197 | Mus musculus thioredoxin mRNA, nuclear gene encoding mitochondrial protein, complete cds {IMAGE [symbol] |
| −2.3 | 462 | 1041 | IMAGE EST {IMAGE:622893} |
| −2.3 | 430 | 996 | ESTs, Weakly similar to RING zinc finger protein [M. musculus ] {IMAGE:922965} |
| −2.3 | 335 | 779 | Mus musculus signal recognition particle receptor beta subunit mRNA, complete cds {IMAGE:482029} |
| −2.3 | 311 | 706 | Mus musculus SH3-containing protein SH3P2 mRNA, partial cds {IMAGE:458781} |
| −2.3 | 261 | 589 | Hormone receptor {IMAGE:641865} |
| −2.3 | 185 | 421 | Heat shock protein, 74 kDa {IMAGE:889543} |
| −2.2 | 904 | 2014 | ESTs, Highly similar to DELTA 1-PYRROLlNE-5-CARBOXYLATE SYNTHETASE [Vigna aconitifolia] {IMA [symbol] |
| −2.2 | 387 | 853 | ESTs {IMAGE:751826} |
| −2.2 | 367 | 807 | IMAGE EST {IMAGE:437685} |
| −2.2 | 366 | 817 | ESTs {IMAGE:350182} |
| −2.2 | 318 | 684 | Metallothionein 1 {IMAGE:480920} |
| −2.2 | 164 | 358 | Mus musculus A10 mRNA, partial cds {IMAGE:333376} |
| −2.1 | 1010 | 2082 | ESTs {IMAGE:479076} |
| −2.1 | 713 | 1474 | Nuclear, factor, erythroid derived 2, like 2 {IMAGE:475505} |
| −2.1 | 640 | 1338 | Metallothionein 1 {IMAGE:480068} |
| −2.1 | 599 | 1257 | M. musculus mRNA for mTGIF protein {IMAGE:722623} |
| −2.1 | 584 | 1235 | Mus musculus asparagine synthetase mRNA, complete cds {IMAGE:337748} |
| −2.1 | 429 | 900 | ESTs {IMAGE:477003} |
| −2.1 | 229 | 473 | IMAGE EST {IMAGE:483649} |
| −2.1 | 135 | 283 | ESTs {IMAGE:637891} |
| −2 | 2153 | 4367 | RAB1, member RAS oncogene family {IMAGE:619501} |
| −2 | 992 | 2008 | ESTs, HIghly similar to HYPOTHETICAL TRP-ASP REPEATS CONTAINING PROTEIN C29E6.01 IN CHR [symbol] |
| −2 | 842 | 1646 | Mus musculus BM28 homolog mRNA, complete cds {IMAGE:441229} |
| −2 | 263 | 533 | IMAGE EST {IMAGE:790122} |
| −2 | 215 | 440 | IMAGE EST {IMAGE:481400} |
| −2 | 101 | 199 | HEMATOPOIETIC CELL PROTEIN-TYROSINE PHOSPHATASE 70Z-PEP {IMAGE:574608} |
| 2 | 10139 | 5129 | Procollagen, type III, alpha 1 {IMAGE:420322} |
| 2 | 2390 | 1169 | Caveolin, caveolae protein, 22 kDa {IMAGE:331186} |
| 2 | 1183 | 583 | ESTs {IMAGE:425777} |
| 2 | 997 | 509 | ESTs, Moderately similar to CYTOCHROME B5, OUTER MITOCHONDRIAL MEMBRANE [Rattus norveg [symbol] |
| 2 | 717 | 363 | Musmusculus L6 antigen mRNA, complete cds {IMAGE:733601} |
| 2 | 643 | 317 | IMAGE EST {IMAGE:437564} |
| 2 | 587 | 287 | IMAGE EST {IMAGE:949532} |
| 2 | 538 | 268 | Carnitine palmitoyltransferase 1, liver {IMAGE:717056} |
| 2 | 494 | 248 | IMAGE EST {IMAGE:477521} |
| 2 | 391 | 199 | Nucleoside phosphorylase {IMAGE:607469} |
| 2 | 322 | 160 | ESTs {IMAGE:847082} |
| 2 | 300 | 148 | ESTs, Highly similar to MITOTIC MAD2 PROTEIN [Saccharomyces cerevisaie] {IMAGE:426406} |
| 2 | 281 | 140 | ESTs {IMAGE:443276} |
| 2 | 278 | 140 | IMAGE EST {IMAGE:465043} |
| 2 | 271 | 135 | ESTs {IMAGE:597249} |
| 2 | 227 | 116 | ESTs {IMAGE:948588} |
| 2 | 210 | 107 | Mus musculus retinoid X receptor interacting protein (RIP14-1No.6) mRNA, complete cds {IMAGE: 7478 [symbol] |
| 2 | 208 | 104 | ESTs, Highly similar to PROTEIN 4.1 [Homo sapiens ] {IMAGE:597748} |
| 2 | 200 | 100 | ESTs, Weakly similar to cDNA EST EMBL:D71941 comes from this gene [C. elegans] {IMAGE:746649} |
| 2 | 165 | 84 | ESTs, Weakly similar to cyclin E [M. musculus] {IMAGE:791957} |
| 2 | 156 | 79 | IMAGE EST {IMAGE:466591} |
| 2 | 148 | 74 | GLUTAMINE SYNTHETASE {IMAGE:693146} |
| 2 | 143 | 71 | ESTs {IMAGE:765727} |
| 2 | 133 | 66 | ESTs {IMAGE:619895} |
| 2.1 | 1848 | 889 | Mus musculus thioredoxin peroxidase (Tpx) mRNA, complete cds {IMAGE:579867} |
| 2.1 | 1817 | 885 | Aquaporin 1 {IMAGE:656654} |
| 2.1 | 1131 | 541 | Caveolin, caveolae protein, 22 kDa {IMAGE:596968} |
| 2.1 | 1013 | 482 | NEURONAL PROTEIN 3.1 {IMAGE:733420} |
| 2.1 | 732 | 347 | Procollagen, type XI, alpha 1 {IMAGE:423028} |
| 2.1 | 586 | 277 | ESTs, Weakly similar to cDNA EST yk486b9.3 comes from this gene [C.elegans] {IMAGE:697835} |
| 2.1 | 402 | 195 | Mus musculus mRNA for mouse rabaptin-5, complete cds {IMAGE:718521} |
| 2.1 | 379 | 179 | IMAGE EST {IMAGE:817962} |
| 2.1 | 363 | 177 | IMAGE EST {IMAGE:658378} |
| 2.1 | 297 | 139 | Mus musculus hematopoietic lineage switch 2 (HLS2) mRNA, complete cds {IMAGE:876463} |
| 2.1 | 285 | 137 | ESTs {IMAGE:330825} |
| 2.1 | 284 | 138 | Farnesyltransferase, CAAX box, alpha {IMAGE:465152] |
| 2.1 | 277 | 135 | IMAGE EST {IMAGE:720096} |
| 2.1 | 275 | 132 | Palmitoyl-protein thioesterase {IMAGE:637934} |
| 2.1 | 259 | 125 | IMAGE EST {IMAGE:408747} |
| 2.1 | 197 | 96 | ESTs, Moderately similar to E1B 19K/Bcl-2-interacting protein Nip3 [H.sapiens] {IMAGE:656945} |
| 2.1 | 177 | 85 | ESTs {IMAGE:837565} |
| 2.1 | 160 | 75 | ESTs {IMAGE:762555} |
| 2.1 | 150 | 70 | IMAGE EST {IMAGE:595978} |
| 2.1 | 103 | 50 | IMAGE EST {IMAGE:350959} |
| 2.2 | 2323 | 1060 | Annexin V {IMAGE:426546} |
| 2.2 | 925 | 421 | ESTs {IMAGE:638302} |
| 2.2 | 865 | 392 | ESTs {IMAGE:891193} |
| 2.2 | 859 | 386 | ESTs, Highly similar to COMPLEMENT C1R COMPONENT PRECURSOR [Homo sapiens ] {IMAGE:61781 [symbol] |
| 2.2 | 720 | 324 | Complement component 2 (within H-2S) {IMAGE:851201} |
| 2.2 | 703 | 316 | Mus musculus transcription factor PBX3b (PBX3b) mRNA, complete cds {IMAGE:425881} |
| 2.2 | 527 | 236 | Uto oncogene homolog {IMAGE:401608} |
| 2.2 | 353 | 159 | IMAGE EST {IMAGE:693565} |
| 2.2 | 337 | 156 | ESTs, Highly similar to glycogen phosphorylase [R. norvegicus] {IMAGE:752290} |
| 2.2 | 305 | 140 | ESTs {IMAGE:596754} |
| 2.2 | 301 | 137 | ESTs {IMAGE:678608} |
| 2.2 | 275 | 125 | ESTs {IMAGE:764398} |
| 2.2 | 217 | 98 | IMAGE EST {IMAGE:922298} |
| 2.2 | 217 | 99 | ESTs {IMAGE:581835} |
| 2.2 | 203 | 94 | ESTs, Weakly similar to cDNA EST EMBL:D71941 comes from this gene [C. elegans] {IMAGE:752290} |
| 2.2 | 200 | 90 | ESTs {IMAGE:679938} |
| 2.2 | 182 | 84 | IMAGE EST {IMAGE:597843} |
| 2.2 | 158 | 73 | ESTs {IMAGE:662476} |
| 2.2 | 154 | 70 | ESTs, Highly similar to HYPOTHETICAL 66.5 KD PROTEIN F02A9.5 IN CHROMOSOME III [Caenorhabdi [symbol] |
| 2.2 | 153 | 70 | IMAGE EST {IMAGE:671280} |
| 2.2 | 142 | 66 | IMAGE EST {IMAGE:463249} |
| 2.2 | 123 | 55 | IMAGE EST {IMAGE:834617} |
| 2.2 | 95 | 44 | ESTs {IMAGE:834572} |
| 2.3 | 1245 | 540 | Dlki-like homolog (Drosophila) {IMAGE:407072} |
| 2.3 | 893 | 393 | IMAGE EST {IMAGE:846536} |
| 2.3 | 812 | 360 | IMAGE EST {IMAGE:425523} |
| 2.3 | 760 | 332 | Alcohol dehydrogenase 1, complex {IMAGE:695105} |
| 2.3 | 494 | 219 | Glycoprotein galactosyltransferase alpha 1, 3 {IMAGE:618535} |
| 2.3 | 355 | 155 | IMAGE EST {IMAGE:583632} |
| 2.3 | 345 | 147 | ESTs, Highly similar to NECDIN [Mus musculus ] {IMAGE:476509} |
| 2.3 | 304 | 132 | ESTs {IMAGE:680450} |
| 2.3 | 211 | 93 | ESTs {IMAGE:576401} |
| 2.3 | 210 | 91 | IMAGE EST {IMAGE:574888} |
| 2.3 | 180 | 79 | ESTs, Highly similar to sorting nexin 1 [H.sapiens] {IMAGE:4568262} |
| 2.3 | 166 | 71 | ESTs {IMAGE:808996} |
| 2.3 | 154 | 68 | ESTs {IMAGE:596030} |
| 2.3 | 136 | 59 | ESTs, Weakly similar to ZINC FINGER PROTEIN HF.12 [Homo sapiens ] {IMAGE:761019} |
| 2.4 | 4099 | 1700 | Tenascin C {IMAGE:736372} |
| 2.4 | 361 | 149 | ESTs, Weakly similar to breast cancer suppressor candidate 1 [H.sapiens] {IMAGE:679316} |
| 2.4 | 341 | 143 | Mus musculus beta-galactoside binding lectin mRNA, complete cds {IMAGE:680815} |
| 2.4 | 298 | 122 | IMAGE EST {IMAGE:693148} |
| 2.4 | 215 | 91 | IMAGE EST {IMAGE:576974} |
| 2.4 | 207 | 87 | ESTs {IMAGE:458996} |
| 2.4 | 194 | 80 | IMAGE EST {IMAGE:733846} |
| 2.4 | 178 | 74 | IMAGE EST {IMAGE:385723} |
| 2.4 | 151 | 63 | ESTs, Moderately similar to E1B 19K/Bcl-2-interacting protein Nip3 [H.sapiens] {IMAGE:464020} |
| 2.5 | 994 | 397 | Mus musculus peroxisomal/mitochondrial dienoyl-CoA isomerase ECH1p (Ech1) mRNA, complete cds [symbol] |
| 2.5 | 381 | 155 | IMAGE EST {IMAGE:775121} |
| 2.5 | 345 | 137 | Mus musculus transcription factor PBX1b (PBX1b) mRNA, complete cds {IMAGE:483688} |
| 2.5 | 315 | 125 | Mast cell growth factor {IMAGE:806850} |
| 2.5 | 239 | 96 | ESTs {IMAGE:401456} |
| 2.5 | 200 | 81 | Carnitine palmitoyltransferase 1, liver {IMAGE:737898} |
| 2.5 | 184 | 73 | IMAGE EST {IMAGE:775154} |
| 2.5 | 173 | 70 | ESTs {IMAGE:618926} |
| 2.5 | 168 | 68 | ESTs {IMAGE:949044} |
| 2.5 | 159 | 63 | IMAGE EST {IMAGE:790674} |
| 2.6 | 1527 | 579 | ESTs, Highly similar to COMPLEMENT C1R COMPONENT PRECURSOR [Homo sapiens ] {IMAGE:72056 [symbol] |
| 2.6 | 555 | 210 | CD9 antigen {IMAGE:421714} |
| 2.6 | 545 | 208 | NEURONAL PROTEIN 3.1 {IMAGE:374970} |
| 2.7 | 3610 | 1352 | Kras oncogene-associated gene {IMAGE:860087} |
| 2.7 | 1160 | 430 | ESTs {IMAGE:850078} |
| 2.7 | 1066 | 394 | ESTs, Weakly similar to tazarotene-induced gene 2 [H.sapiens] {IMAGE:695491} |
| 2.7 | 213 | 79 | ESTs {IMAGE:467873} |
| 2.8 | 698 | 248 | ESTs, Highly similar to ACETYL-COA ACETYLTRANSFERASE PRECURSOR, MITOCHONDRIAL [Homo [symbol] |
| 2.8 | 296 | 104 | ESTs, Highly similar to HYPOTHETICAL 47.9 KD PROTEIN B0303.3 IN CHROMOSOME III [Ceanorhabdi [symbol] |
| 2.8 | 203 | 73 | IMAGE EST {IMAGE:670393} |
| 2.9 | 1983 | 675 | Cystatin 3 {IMAGE:402614} |
| 2.9 | 271 | 95 | IMAGE EST {IMAGE:949656} |
| 3 | 727 | 244 | Malate dehydrogenase, soluble {IMAGE:318346} |
| 3.1 | 597 | 194 | Mus musculus LIM protein 3 (mSLIM3) mRNA, complete cds {IMAGE:457264} |
| 3.2 | 738 | 228 | ESTs, Highly similar to DERMATOPONTIN [Bos taurus] {IMAGE:330218} |
| 3.2 | 529 | 166 | Adenylate cyclase 7 {IMAGE:387280} |
| 3.3 | 1545 | 473 | Tissue inhibitor of metalloproteinase 2 {IMAGE:902923} |
| 3.3 | 558 | 171 | ESTs, Highly similar to DESTRIN [Homo sapiens ; Sus scrofa] {IMAGE:335112} |
| 3.3 | 351 | 106 | IMAGE EST {IMAGE:876446} |
| 3.3 | 247 | 75 | IMAGE EST {IMAGE:920211} |
| 3.4 | 1612 | 479 | IMAGE EST {IMAGE:374228} |
| 3.4 | 589 | 173 | Erythrocyte protein band 4.1 {IMAGE:444037} |
| 3.7 | 2263 | 609 | Tissue inhibitor of metalloproeinase 2 {IMAGE:831964} |
| 4.1 | 1504 | 370 | ESTs, Moderately similar to COMPLEMENT C1S COMPONENT PRECURSOR [H. sapiens ] {IMAGE:67617 [symbol] |

TABLE 2
64 common genes upregulated by FSH, 024 & forskolin

| | | |
|---|---|---|
| Antigen identified by monoclonal antibodies 4F2 {IMAGE:478301} | 478301 | AA049696.1 |
| Chaperonin subunit 4 (delta) {IMAGE:459668} | 459668 | AA027583.1 |
| ESTs {IMAGE:350182} | 350182 | W34722.1 |
| ESTs {IMAGE:424848} | 424848 | W98118.1 |
| ESTs {IMAGE:426033} | 426033 | AA002836.1 |
| ESTs {IMAGE:427480} | 427480 | AA002452.1 |
| ESTs {IMAGE:477003} | 477003 | AA048121.1 |
| ESTs {IMAGE:482641} | 482641 | AA061982.1 |
| ESTs {IMAGE:483476} | 483476 | AA060036.1 |
| ESTs {IMAGE:598824} | 598824 | AA168416.1 |
| ESTs {IMAGE:640085} | 640085 | AA198542.1 |
| ESTs {IMAGE:656089} | 656089 | AA239554.1 |
| ESTs {IMAGE:680250} | 680250 | AA237600.1 |
| ESTs {IMAGE:749313} | 749313 | AA288555.1 |
| ESTs {IMAGE:818790} | 818790 | AA467382.1 |
| ESTs {IMAGE:876063} | 876063 | AA475435.1 |
| ESTs, Highly similar to CYSTATHIONINE GAMMA-LYASE [Homo [symbol] | 525119 | AA096870.1 |
| ESTs, Highly similar to CYSTATHIONINE GAMMA-LYASE [Homo [symbol] | 679235 | AA245993.1 |
| ESTs, Highly similar to REPLICATION PROTEIN A 14 KD SUBUN [symbol] | 425703 | AA000318.1 |
| ESTs, Highly similar to SERINE HYDROXYMETHYLTRANSFERA [symbol] | 676311 | AA208877.1 |
| ESTs, Highly similar to (defline not available 4929631) [H. sapiens [symbol] | 467379 | AA036624.1 |
| ESTs, Moderately similar to tumorous imaginal discs protein Tid [symbol] | 478167 | AA049615.1 |
| ESTs, Weakly similar to (defline not available 3874389) [C. elegan [symbol] | 334182 | W16247.1 |
| ESTs, Weakly similar to GARP PROTEIN PRECURSOR [H. sapiens [symbol] | 479076 | AA048874.1 |
| ESTs, Weakly similar to HYPOTHETICAL 11.4 KD PROTEIN C13G [symbol] | 579733 | AA116946.1 |
| ESTs, Weakly similar to ORF YGL231c [S. cerevisiae] {IMAGE:442 [symbol] | 442681 | AA015149.1 |
| ESTs, Weakly similar to putative [C. elegans] {IMAGE:571422} | 571422 | AA109015.1 |
| ESTs, Weakly similar to RING zinc finger protein [M. musculus] {I [symbol] | 922965 | AA511365.1 |
| ESTs, Weakly similar to similar to EF hand domains [C. elegans] { [symbol] | 337667 | W18735.1 |
| Extracellular matrix protein 1 {IMAGE:874833} | 874833 | AA474897.1 |
| GALECTIN-3 {IMAGE:717226} | 717226 | AA403841.1 |
| Glutamate oxaloacetate transminase 1, soluble {IMAGE:481381} | 481381 | AA060494.1 |
| Glutathione-S-transferase, alpha 3 {IMAGE:766582} | 766582 | AA274682.1 |
| Growth arrest specific 2 {IMAGE:820540} | 820540 | AA423395.1 |
| HEMATOPOIETIC CELL PROTEIN-TYROSINE PHOSPHATASE 70 [symbol] | 574608 | AI323214.1 |
| Heme oxygenase (decycling) 1 {IMAGE:677499} | 677499 | AA213167.1 |
| Hormone receptor {IMAGE:439773} | 439773 | AA008625.1 |
| IMAGE EST {IMAGE:315676} | 315676 | W09957.1 |
| IMAGE EST {IMAGE:318157} | 318157 | W11665.1 |
| IMAGE EST {IMAGE:330146} | 330146 | W11535.1 |
| IMAGE EST {IMAGE:367445} | 367445 | W50706.1 |
| IMAGE EST {IMAGE:437685} | 437685 | AA007828.1 |
| IMAGE EST {IMAGE:466678} | 466678 | AA031159.1 |
| IMAGE EST {IMAGE:467785} | 467785 | AA036495.1 |
| IMAGE EST {IMAGE:479247} | 479247 | AA048730.1 |
| IMAGE EST {IMAGE:480920} | 480920 | AA064247.1 |
| IMAGE EST {IMAGE:483649} | 483649 | AA061366.1 |
| IMAGE EST {IMAGE:622893} | 622893 | AA177702.1 |
| IMAGE EST {IMAGE:635746} | 635746 | AA166372.1 |
| IMAGE EST {IMAGE:681424} | 681424 | AA237757.1 |
| IMAGE EST {IMAGE:790122} | 790122 | AA387971.1 |
| IMAGE EST {IMAGE:874030} | 874030 | AA472200.1 |
| Lymphocyte antigen 6 complex {IMAGE:580715} | 580715 | AA145865.1 |
| Mouse mRNA for dbpA murine homologue, complete cds {IMAGE [symbol] | 481949 | AA059953.1 |
| Mus musculus A10 mRNA, partial cds {IMAGE:333376} | 333376 | W15888.1 |
| Mus musculus A10 mRNA, partial cds {IMAGE:385441} | 385441 | W61383.1 |
| Mus musculus elF-1A (elF-1A) mRNA, complete cds {IMAGE:7473 [symbol] | 747322 | AA274946.1 |
| Mus musculus protein kinase C inhibitor (mPKCI) mRNA, comple [symbol] | 533117 | AA068901.1 |
| Mus musculus secreted carbonic anhydrase isozyme VI precurso [symbol] | 329940 | AI327498.1 |
| Mus musculus SH3-containing protein SH3P2 mRNA, partial cds [symbol] | 458781 | AA024088.1 |
| MYELOID DIFFERENTIATION PRIMARY RESPONSE PROTEIN MY [symbol] | 475803 | AA050417.1 |
| Nuclear, factor, erythroid derived 2, like 2 {IMAGE:475505} | 475505 | AA044475.1 |
| T-complex testis expressed 1 {IMAGE:762306} | 762306 | AA277421.1 |
| TRYPTOPHANYL-TRNA SYNTHETASE {IMAGE:367765} | 367765 | W53959.1 |

TABLE 3
024 16 hr time point 121 genes upregulated > = 2-fold 0226AAMG

| Fold | Cont | 24 | Gene Name | Acc # |
|---|---|---|---|---|
| 2.7 | 1331 | 3587 | antigen identified by monoclonal antibodies 4F2 {IMAGE:478301} | AA049696.1 |
| 5.1 | 136 | 694 | carbonic anhydrase 6 {IMAGE:329940} | AI327498.1 |
| 2.2 | 165 | 366 | CD39 antigen-like 4 {IMAGE:574894} | AA120757.1 |
| 2.6 | 213 | 564 | chaperonin subunit 4 (delta) {IMAGE:459668} | AA027583.1 |
| 2.3 | 161 | 367 | E26 avian leukemia oncogene 2, 3′ domain {IMAGE:949055} | AA543913.1 |
| 2.7 | 486 | 1303 | ESTs {IMAGE:315676} | W09957.1 |
| 3.3 | 577 | 1914 | ESTs {IMAGE:350182} | W34722.1 |
| 2.1 | 336 | 689 | ESTs {IMAGE:367445} | W50706.1 |
| 2 | 408 | 800 | ESTs {IMAGE:386218} | W65070.1 |
| 2.2 | 479 | 1066 | ESTs {IMAGE:404057} | W82577.1 |
| 2.3 | 558 | 1305 | ESTs {IMAGE:419146} | W88005.1 |
| 2.1 | 290 | 597 | ESTs {IMAGE:421524} | W97155.1 |
| 2.6 | 89 | 233 | ESTs {IMAGE:424848} | W98118.1 |
| 3.3 | 241 | 786 | ESTs {IMAGE:426033} | AA002836.1 |
| 2.4 | 93 | 219 | ESTs {IMAGE:427480} | AA002452.1 |
| 2.4 | 296 | 699 | ESTs {IMAGE:436999} | AA002783.1 |
| 2.1 | 187 | 392 | ESTs {IMAGE:439411} | AA004111.1 |
| 2.2 | 329 | 726 | ESTs {IMAGE:466678} | AA031159.1 |
| 2.7 | 236 | 643 | ESTs {IMAGE:477003} | AA048121.1 |
| 2.5 | 153 | 378 | ESTs {IMAGE:479247} | AA048730.1 |
| 2.3 | 176 | 403 | ESTs {IMAGE:479913} | AA051561.1 |
| 2 | 71 | 139 | ESTs {IMAGE:480197} | AA058059.1 |
| 4.8 | 287 | 1384 | ESTs {IMAGE:482641} | AA061982.1 |
| 2.1 | 339 | 725 | ESTs {IMAGE:483476} | AA060036.1 |
| 2 | 283 | 579 | ESTs {IMAGE:483649} | AA061366.1 |
| 5.3 | 62 | 327 | ESTs {IMAGE:598824} | AA168416.1 |
| 2.3 | 236 | 553 | ESTs {IMAGE:618611} | AA174941.1 |
| 3.3 | 98 | 321 | ESTs {IMAGE:620209} | AA177920.1 |
| 2.4 | 247 | 588 | ESTs {IMAGE:622893} | AA177702.1 |
| 2.2 | 144 | 319 | ESTs {IMAGE:635810} | AA185313.1 |
| 3.1 | 141 | 442 | ESTs {IMAGE:640085} | AA198542.1 |
| 3.4 | 339 | 1168 | ESTs {IMAGE:656089} | AA239554.1 |
| 2.4 | 256 | 607 | ESTs {IMAGE:680250} | AA237600.1 |
| 2 | 356 | 714 | ESTs {IMAGE:722625} | AA260445.1 |
| 2 | 132 | 264 | ESTs {IMAGE:746599} | AA239554.1 |
| 2.4 | 91 | 219 | ESTs {IMAGE:749313} | AA268055.1 |
| 2 | 235 | 480 | ESTs {IMAGE:751826} | AA288555.1 |
| 2.2 | 169 | 373 | ESTs {IMAGE:762306} | AA396152.1 |
| 2.6 | 176 | 456 | ESTs {IMAGE:790122} | AA277421.1 |
| 2.6 | 101 | 263 | ESTs {IMAGE:818790} | AA387971.1 |
| 2.1 | 131 | 270 | ESTs {IMAGE:876063} | AA467382.1 |
| 2 | 103 | 201 | ESTs {IMAGE:876106} | AA475435.1 |
| 5.1 | 200 | 1029 | ESTs, Highly similar to CYSTATHIONINE GAMMA-LYASE [Homo sapien [symbol] | AA096870.1 |
| 4.2 | 90 | 374 | ESTs, Highly similar to CYSTATHIONINE GAMMA-LYASE [Homo sapien [symbol] | AA245993.1 |
| 2.4 | 551 | 1318 | ESTs, Highly similar to PUTATIVE ASPARAGINYL-TRNA SYNTHETASE [symbol] | AA399854.1 |
| 2.3 | 253 | 581 | ESTs, Highly similar to REPLICATION PROTEIN A 14 KD SUBUNIT [Hom [symbol] | AA000318.1 |
| 2 | 432 | 862 | ESTs, Highly similar to SERINE HYDROXYMETHYLTRANSFERASE, MIT [symbol] | AA208877.1 |
| 2.2 | 534 | 1166 | ESTs, Highly similar to CGI-BI protein [H. sapiens] {IMAGE:467379} | AA036624.1 |
| 2.1 | 160 | 331 | ESTs, Highly similar to db83 [R. norvegicus] {IMAGE:737629} | AA277149.1 |
| 2.1 | 259 | 538 | ESTs, Highly similar to exportin t [H. sapiens] {IMAGE:639640} | AA197461.1 |
| 2.2 | 353 | 784 | ESTs, Highly similar to pEachy [R. norvegicus] {IMAGE:747789} | AA274849.1 |
| 2.5 | 517 | 1293 | ESTs, Highly similar to probable calcium-binding protein [H. sapiens] {IM [symbol] | W18735.1 |
| 2.2. | 328 | 726 | ESTs, Moderately similar to CHLORINE CHANNEL PROTEIN P64 [Bos ta [symbol] | AA466508.1 |
| 2.3 | 313 | 717 | ESTs, Moderately similar to Unknown [H. sapiens] {IMAGE:764677} | AA273209.1 |
| 2.3 | 256 | 586 | ESTs, Weakly similar to (defline not available 5852158) [M. musculus] {IM [symbol] | AA275027.1 |
| 2.1 | 157 | 334 | ESTs, Weakly similar to 3-OXOACYL-[ACYL-CARRlER PROTEIN] REDUC [symbol] | W65003.1 |
| 2.7 | 436 | 1198 | ESTs, Weakly similar to ACTIN POLYMERIZATION INHIBITOR [Gallus ga [symbol] | AA003272.1 |
| 2.1 | 165 | 339 | ESTs, Weakly similar to cDNA EST EMBL:D70402 comes from this gene [symbol] | AA120013.1 |
| 3.3 | 532 | 1752 | ESTs, Weakly similar to cDNA EST EMBL:D75506 comes from this gene [symbol] | W16247.1 |
| 2 | 461 | 915 | ESTs, Weakly similar to coded for by C. elegans cDNA yk157f8.5 [C. eleg [symbol] | W43938.1 |
| 2.1 | 948 | 1978 | ESTs, Weakly similar to GARP PROTEIN PRECURSOR [H. sapiens] {IMA [symbol] | AA048874.1 |
| 2.6 | 225 | 591 | ESTs, Weakly similar to heat schock protein hsp40-3 [M. musculus] {IMA [symbol] | AA049615.1 |
| 2.2 | 248 | 546 | ESTs, Weakly similar to HYPOTHETICAL 11.4 KD PROTEIN C13G6.04 IN [symbol] | W11535.1 |
| 2.6 | 124 | 327 | ESTs, Weakly similar to HYPOTHETICAL 11.4 KD PROTEIN C13G6.04 IN [symbol] | AA116946.1 |
| 2 | 454 | 887 | ESTs, Weakly similar to INTERFERON-INDUCED PROTEIN 6-16 PRECUR [symbol] | W99140.1 |
| 2 | 366 | 732 | ESTs, Weakly similar to LYMPHOCYTE ANTIGEN LY-6A.2/LY-6E.1 PREC [symbol] | AA472994.1 |
| 2.1 | 166 | 343 | ESTs, Weakly similar to ORF YGL231c [S. cerevisiae] {IMAGE:442681} | AA015149.1 |
| 2 | 232 | 469 | ESTs, Weakly similar to putative [C. elegans] {IMAGE:571422} | AA109015.1 |
| 2.2 | 863 | 1882 | ESTs, Weakly similar to SIK similar protein [M. musculus] {IMAGE:93349 [symbol] | AA542348.1 |
| 2 | 504 | 1006 | ESTs, Weakly similar to similar to leucyl-tRNA synthetase [C. elegans] {I [symbol] | W11665.1 |
| 3.4 | 203 | 681 | extracellular matrix protein 1 {IMAGE:678765} | AA237378.1 |
| 4.7 | 417 | 1960 | extracellular matrix protein 1 {IMAGE:874833} | AA474897.1 |
| 2 | 205 | 412 | Fyn proto-oncogene {IMAGE:385072} | W62969.1 |
| 2.8 | 657 | 1835 | glutamate oxaloacetate transaminase 1, soluble {IMAGE:481381} | AA060494.1 |
| 2.8 | 256 | 718 | glutathione-S-transferase, alpha 3 {IMAGE:766582} | AA274682.1 |
| 3.5 | 486 | 1713 | glutathione-S-tranSferase, alpha 4 {IMAGE:367627) | W54349.1 |
| 2.2 | 151 | 330 | growth arrest specific 2 {IMAGE:820540} | AA423395.1 |
| 2.2 | 1082 | 2398 | heat shock protein, 74 kDa, A {IMAGE:444027} | AA014915.1 |
| 3.4 | 189 | 636 | heme oxygenase (decycling) 1 {IMAGE:677499} | AA213167.1 |
| 2 | 472 | 938 | histidine triad nucleotide-binding protein {IMAGE:533117} | AA068901.1 |
| 2.3 | 523 | 1190 | homeo box B9 {IMAGE:422746} | W97853.1 |
| 2.4 | 253 | 604 | hormone receptor {IMAGE:439773} | AA008625.1 |
| 2.2 | 114 | 250 | hormone receptor {IMAGE:641865} | AA209882.1 |
| 2.1 | 649 | 1345 | HS1 binding protein {IMAGE:874591} | AA472437.1 |
| 2.3 | 469 | 1056 | lectin, galactose binding, soluble 3 {IMAGE:717226} | AA403841.1 |
| 2.4 | 785 | 1868 | lymphocyte antigen 6 complex {IMAGE:580715} | AA145865.1 |
| 2.5 | 285 | 724 | lymphocyte antigen 6 complex, locus C {IMAGE:425855} | AA000712.1 |
| 2 | 205 | 410 | metallothionein 1 {IMAGE:480920} | AA064247.1 |
| 2 | 548 | 1083 | metallothionein 2 {IMAGE:643725} | AA203775.1 |
| 2.2 | 1035 | 2244 | methylenetetrahydrofolate dehydrogenase (NAD + dependent), methenyl [symbol] | W84014.1 |
| 2.8 | 335 | 954 | Mouse mRNA for dbpA murine homologue, complete cds {IMAGE:48194 [symbol] | AA059953.1 |
| 2.6 | 277 | 719 | Mus musculus A10 mRNA, partial cds {IMAGE:333376} | W15888.1 |
| 2.5 | 509 | 1296 | Mus musculus A10 mRNA, partial cds {IMAGE:385441} | W61383.1 |
| 2.8 | 821 | 2317 | Mus musculus asparigine synthetase mRNA, complete cds {IMAGE:337 [symbol] | W29492.1 |
| 2.1 | 239 | 512 | Mus musculus elF-1A (elF-1A) mRNA, complete cds {IMAGE:351631} | W41459.1 |
| 2.1 | 663 | 1420 | Mus musculus elF-1A (elF-1A) mRNA, complete cds {IMAGE:747322} | AA274946.1 |
| 2.9 | 295 | 841 | Mus musculus hsp40 mRNA for heat shock protein 40, complete cds {IM [symbol] | W75670.1 |
| 2.7 | 340 | 902 | Mus musculus mRNA for Sid1669p, complete cds {IMAGE:922965} | AA511365.1 |
| 2.2 | 248 | 538 | Mus musculus SH3-containing protein SH3P2 mRNA, partial cds {IMAGE [symbol] | AA024088.1 |
| 2.1 | 252 | 528 | Mus musculus SH3-containing protein SH3P2 mRNA, partial cds {IMAGE [symbol] | AA166372.1 |
| 2.3 | 246 | 559 | Mus musculus thioredoxin mRNA, nuclear gene encoding mitochondrial [symbol] | AA242573.1 |
| 2.1 | 620 | 1324 | myelocytomatosis oncogene {IMAGE:441346} | AA009268.1 |
| 11.7 | 265 | 3097 | myeloid differentiation primary response gene 116 {IMAGE:475803} | AA050417.1 |
| 2.3 | 512 | 1154 | myosin, heavy polypeptide 8, skeletal muscle, perinatal {IMAGE:317476} | W13528.1 |
| 2.3 | 307 | 693 | nuclear factor of activated T-cells, cytoplasmic 2 {IMAGE:904738} | AA521764.1 |
| 2.4 | 475 | 1126 | nuclear, factor, erythroid derived 2, like 2 {IMAGE:475505} | AA044475.1 |
| 2 | 231 | 457 | prion protein {IMAGE:421749} | W99102.1 |
| 2.9 | 68 | 197 | protein tyrosine phosphatase, non-receptor type 8 {IMAGE:574608} | AI323214.1 |
| 2.3 | 170 | 397 | Public domain EST {IMAGE:437685} | AA007828.1 |
| 2 | 153 | 301 | Public domain EST {IMAGE:439033} | AA008240.1 |
| 4.1 | 179 | 740 | Public domain EST {IMAGE:467785} | AA036495.1 |
| 2.2 | 267 | 596 | Public domain EST {IMAGE:481400} | AA060500.1 |
| 2 | 312 | 636 | Public domain EST {IMAGE:639704} | AA197393.1 |
| 2.1 | 80 | 166 | Public domain EST {IMAGE:641223} | AA200448.1 |
| 2.2 | 186 | 403 | Public domain EST {IMAGE:681424} | AA237757.1 |
| 2 | 208 | 412 | Public domain EST {IMAGE:694065} | AA243954.1 |
| 2.1 | 174 | 362 | Public domain EST {IMAGE:733734} | AA272876.1 |
| 2.1 | 341 | 700 | Public domain EST {IMAGE:763628} | AA285580.1 |
| 2.1 | 398 | 848 | Public domain EST {IMAGE:874030} | AA472200.1 |
| 2.2 | 376 | 820 | stimulated by retinoic acid 14 {IMAGE:480896} | AA064241.1 |
| 2.7 | 192 | 520 | tryptophanyl-tRNA synthetase {IMAGE:367765} | W53959.1 |

TABLE 4
Forskolin 16 hr time point 106 genes upregulated > = 2-fold 022DAAMH

| Fold | Cont | Forsk | Gene name | Acc # |
|---|---|---|---|---|
| 2.1 | 1661 | 3449 | antigen identified by monoclonal antibodies 4F2 {IMAGE:478301} | AA049696.1 |
| 4.1 | 183 | 750 | carbonic anhydrase 6 {IMAGE:329940} | AI327498.1 |
| 2.4 | 227 | 552 | chaperonin subunit 4 (delta) {IMAGE:459668} | AA027583.1 |
| 2.1 | 259 | 545 | chondroitin sulfate proteoglycan 2 {IMAGE:355990} | W49048.1 |
| 2.1 | 624 | 1320 | ESTs {IMAGE:315676} | W09957.1 |
| 2 | 424 | 859 | ESTs {IMAGE:316914} | W11926.1 |
| 2 | 939 | 1885 | ESTs {IMAGE:317466} | W34061.1 |
| 3.7 | 448 | 1660 | ESTs {IMAGE:350182} | W34722.1 |
| 2.7 | 374 | 1004 | ESTs {IMAGE:367445} | W50706.1 |
| 2.3 | 512 | 1203 | ESTs {IMAGE:372421} | W53621.1 |
| 2 | 396 | 783 | ESTs {IMAGE:386218} | W65070.1 |
| 2 | 85 | 169 | ESTs {IMAGE:403166} | W82868.1 |
| 2 | 378 | 769 | ESTs {IMAGE:404057} | W82577.1 |
| 2.5 | 106 | 270 | ESTs {IMAGE:424848} | W98118.1 |
| 3.8 | 303 | 1150 | ESTs {IMAGE:426033} | AA002836.1 |
| 2.4 | 140 | 339 | ESTs {IMAGE:427480} | AA002452.1 |
| 2.1 | 365 | 761 | ESTs {IMAGE:466678} | AA031159.1 |
| 2.8 | 272 | 757 | ESTs {IMAGE:477003} | AA048121.1 |
| 2.3 | 154 | 352 | ESTs {IMAGE:479247} | AA048730.1 |
| 6.2 | 209 | 1295 | ESTs {IMAGE:482641} | AA061982.1 |
| 2.2 | 387 | 834 | ESTs {IMAGE:483476} | AA060036.1 |
| 2.3 | 310 | 709 | ESTs {IMAGE:483649} | AA061366.1 |
| 2.3 | 374 | 859 | ESTs {IMAGE:572819} | AA110791.1 |
| 8.6 | 58 | 498 | ESTs {IMAGE:598824} | AA168416.1 |
| 2.5 | 101 | 251 | ESTs {IMAGE:620209} | AA177920.1 |
| 2.1 | 246 | 527 | ESTs {IMAGE:622893} | AA177702.1 |
| 2 | 238 | 469 | ESTs {IMAGE:636730} | AA189425.1 |
| 2.5 | 114 | 289 | ESTs {IMAGE:640085} | AA198542.1 |
| 3.8 | 324 | 1223 | ESTs {IMAGE:656089} | AA239554.1 |
| 2.2 | 258 | 564 | ESTs {IMAGE:680250} | AA237600.1 |
| 2.7 | 52 | 139 | ESTs {IMAGE:749313} | AA288555.1 |
| 2 | 121 | 236 | ESTs {IMAGE:762306} | AA277421.1 |
| 2.7 | 226 | 607 | ESTs {IMAGE:790122} | AA387971.1 |
| 4.7 | 59 | 278 | ESTs {IMAGE:818790} | AA467382.1 |
| 2.3 | 120 | 274 | ESTs {IMAGE:876063} | AA475435.1 |
| 4 | 149 | 602 | ESTs, Highly similar to CYSTATHIONINE GAMMA-LYASE [Homo sap [symbol] | AA096870.1 |
| 3.4 | 52 | 175 | ESTs, Highly similar to CYSTATHIONINE GAMMA-LYASE [Homo sap [symbol] | AA245993.1 |
| 2 | 683 | 1346 | ESTs, Highly similar to DELTA 1-PYRROLINE-5-CARBOXYLATE SYN [symbol] | W41878.1 |
| 2.7 | 200 | 531 | ESTs, Highly similar to GLYPICAN-3 PRECURSOR [Rattus norvegicu [symbol] | AA274932.1 |
| 2.1 | 506 | 1072 | ESTs, Highly similar to HAM1 PROTEIN [Saccharomyces cervisiae] [symbol] | W34474.1 |
| 2 | 372 | 761 | ESTs, Highly similar to HYPOTHETICAL 25.7 KD PROTEIN IN MSH1- [symbol] | W54688.1 |
| 2.3 | 311 | 708 | ESTs, Highly similar to REPLICATION PROTEIN A 14 KD SUBUNIT [H [symbol] | AA000318.1 |
| 2 | 304 | 602 | ESTs, Highly similar to SERINE HYDROXYMETHYLTRANSFERASE, [symbol] | AA208877.1 |
| 11.6 | 916 | 10602 | ESTs, Highly similar to acid ceramidase [M. musculus] {IMAGE:76308 [symbol] | AA286605.1 |
| 2.1 | 574 | 1206 | ESTs, Highly similar to CGI-81 protein [H. sapiens] {IMAGE:467379} | AA036624.1 |
| 2 | 224 | 443 | ESTs, Highly similar to HSPC040 protein [H. sapiens] {IMAGE:332442} | W08432.1 |
| 2.1 | 657 | 1358 | ESTs, Highly similar to probable calcium-binding protein [H. sapiens] [symbol] | W18735.1 |
| 2.2 | 377 | 828 | ESTs, Highly similar to similar to Schizosaccharomyces pombe splic [symbol] | W11916.1 |
| 2.1 | 359 | 744 | ESTs, Moderately similar to NADH-UBIQUINONE OXIDOREDUCTASE [symbol] | W97248.1 |
| 3.2 | 715 | 2301 | ESTs, Weakly similar to cDNA EST EMBL:D75506 comes from this ge [symbol] | W16247.1 |
| 2.2 | 658 | 1423 | ESTs, Weakly similar to GARP PROTEIN PRECURSOR [H. sapiens] {IM [symbol] | AA048874.1 |
| 2.6 | 245 | 642 | ESTs, Weakly similar to heat shock protein hsp40-3 [M. musculus] {IM [symbol] | AA049615.1 |
| 2.7 | 1071 | 2916 | ESTs, Weakly similar to HISTIDINE-RICH PROTEIN KE4 [M. musculus] [symbol] | W18585.1 |
| 2.5 | 218 | 551 | ESTs, Weakly similar to HYPOTHETICAL 11.4 KD PROTEIN C13G6.04 [symbol] | W11535.1 |
| 2 | 161 | 330 | ESTs, Weakly similar to HYPOTHETICAL 11.4 KD PROTEIN C13G6.04 [symbol] | AA116946.1 |
| 2.3 | 300 | 698 | ESTs, Weakly similar to LYMPHOCYTE ANTIGEN LY-6A.2/LY-6E.1 PR [symbol] | AA472994.1 |
| 2.1 | 124 | 263 | ESTs, Weakly similar to mCAC [M. musculus] {IMAGE:350881} | W40994.1 |
| 2.5 | 212 | 536 | ESTs, Weakly similar to ORF YGL231c [S. cerevisiae] {IMAGE:442681} | AA015149.1 |
| 2 | 177 | 359 | ESTs, Weakly similar to putative [C. elegans] {IMAGE:571422} | AA109015.1 |
| 2.3 | 724 | 1644 | ESTs, Weakly similar to similar to leucyl-tRNA synthetase [C. elegans] | W11665.1 |
| 2.9 | 1092 | 3190 | ESTs, Weakly similar to similar to nucleotide translocator [C. elegans] | AA213247.1 |
| 3.7 | 193 | 721 | extracellular matrix protein 1 {IMAGE:678765} | AA237378.1 |
| 7.4 | 369 | 2744 | extracellular matrix protein 1 {IMAGE:874833} | AA474897.1 |
| 2.9 | 497 | 1443 | glutamate oxaloacetate transaminase 1, soluble {IMAGE:481381} | AA060494.1 |
| 3 | 254 | 755 | glutathione-S-transferase, alpha 3 {IMAGE:766582} | AA274682.1 |
| 2.7 | 427 | 1169 | glutathione-S-transferase, alpha 4 {IMAGE:367627} | W54349.1 |
| 3 | 108 | 326 | growth arrest specific 2 {IMAGE:820540} | AA423395.1 |
| 3.2 | 198 | 627 | heme oxygenase (decycling) 1 {IMAGE:677499} | AA213167.1 |
| 2 | 291 | 570 | histidine triad nucleotide-binding protein {IMAGE:533117} | AA068901.1 |
| 2.3 | 283 | 658 | hormone receptor {IMAGE:439773} | AA008625.1 |
| 2 | 87 | 174 | hormone receptor {IMAGE:641865} | AA209882.1 |
| 2.6 | 482 | 1263 | lectin, galactose binding, soluble 3 {IMAGE:717226} | AA403841.1 |
| 3 | 608 | 1820 | lymphocyte antigen 6 complex {IMAGE:580715} | AA145865.1 |
| 3.6 | 371 | 1333 | lymphocyte antigen 6 complex, locus C {IMAGE:425855} | AA000712.1 |
| 2.1 | 312 | 640 | male enhanced antigen 1 {IMAGE:463746} | AA028786.1 |
| 2.4 | 435 | 1058 | metallothionein 1 {IMAGE:480068} | AA051654.1 |
| 2.6 | 183 | 475 | metallothionein 1 {IMAGE:480920} | AA064247.1 |
| 2.2 | 324 | 718 | Mouse chromatin nonhistone high mobility group protein (HGM-I(Y), [symbol] | AA538243.1 |
| 2.2 | 463 | 1033 | Mouse mRNA for dbpA murine homologue, complete cds {IMAGE:481 [symbol] | AA059953.1 |
| 2 | 219 | 428 | Mus musculus A10 mRNA, partial cds {IMAGE:333376} | W15888.1 |
| 2.1 | 673 | 1403 | Mus musculus A10 mRNA, partial cds {IMAGE:385441} | W61383.1 |
| 2 | 646 | 1292 | Mus musculus elF-1A (elF-1A) mRNA, complete cds {IMAGE:747322} | AA274946.1 |
| 2.2 | 363 | 786 | Mus musculus mRNA for HIRA-interacting protein (HIRIP5) {IMAGE:7 [symbol] | AA270607.1 |
| 2.6 | 344 | 878 | Mus musculus mRNA for Sid1669p, complete cds {IMAGE:922965} | AA511365.1 |
| 2.9 | 253 | 730 | Mus musculus SH3-containing protein SH3P2 mRNA, partial cds {IMA [symbol] | AA024088.1 |
| 2.5 | 148 | 372 | Mus musculus SH3-containing protein SH3P2 mRNA, partial cds {IMA [symbol] | AA166372.1 |
| 10.6 | 268 | 2833 | myeloid differentiation primary response gene 116 {IMAGE:475803} | AA050417.1 |
| 2.3 | 652 | 1470 | myosin, heavy polypeptide 8, skeletal muscle, perinatal {IMAGE:3174 [symbol] | W13528.1 |
| 2 | 355 | 720 | nuclear, factor, erythroid derived 2, like 2 {IMAGE:475505} | AA044475.1 |
| 2 | 309 | 625 | periplakin {IMAGE:571984} | AA105152.1 |
| 2 | 65 | 128 | protein kinase C, delta {IMAGE:421002} | W91539.1 |
| 2.4 | 75 | 180 | protein tyrosine phosphatase, non-receptor type 8 {IMAGE:574608} | AI323214.1 |
| 2.2 | 122 | 265 | Public domain EST {IMAGE:426378} | AA002886.1 |
| 2.9 | 192 | 549 | Public domain EST {IMAGE:437685} | AA007828.1 |
| 7 | 184 | 1293 | Public domain EST {IMAGE:467785} | AA036495.1 |
| 2.3 | 108 | 246 | Public domain EST {IMAGE:574227} | AA119136.1 |
| 2.2 | 145 | 317 | Public domain EST {IMAGE:681424} | AA237757.1 |
| 3.2 | 69 | 223 | Public domain EST {IMAGE:716713} | AA265198.1 |
| 2 | 116 | 237 | Public domain EST {IMAGE:805306} | AA473329.1 |
| 2.3 | 337 | 765 | Public domain EST {IMAGE:874030} | AA472200.1 |
| 2 | 1049 | 2049 | requiem {IMAGE:573346} | AI323194.1 |
| 2.4 | 127 | 304 | sphingosine kinase 1 {IMAGE:425961} | AA000819.1 |
| 2 | 839 | 1677 | split hand/foot deleted gene 1 {IMAGE:850971} | AA462396.1 |
| 2.4 | 365 | 894 | stimulated by retinoic acid 14 {IMAGE:480896} | AA064241.1 |
| 2.1 | 256 | 530 | TG interacting factor {IMAGE:722623} | AA260654.1 |
| 3.4 | 227 | 761 | tryptophanyl-tRNA synthetase {IMAGE:367765} | W53959.1 |

We claim:

1. A method for identifying an agent which modulates an FSH or FSH Mimetic influenced cellular process or response, the method comprising:
a) exposing a sample of cells to FSH or FSH Mimetic;
b) determining the level of expression in the sample of cells of one or more FSH OR FSH Mimetic stimulated genes (Tables 1, 2, 3) in the presence and absence of a selected agent; and
c) identifying that the agent modulates an FSH or FSH Mimetic influenced cellular process or response when the expression of the one or more FSH or FSH Mimetic stimulated genes in the cell sample in the presence of the agent differs from the expression of the one or more FSH or FSH Mimetic stimulated genes in the absence of the agent.

2. A method for identifying an agent which modulates an FSH or FSH Mimetic influenced cellular process or response, the method comprising:
a) exposing a sample of cells to FSH or FSH Mimetic;
b) determining the activity in the sample of cells of the product of one or more FSH or FSH Mimetic stimulated genes (Table 1, 2, or 3) in the presence and absence of a selected agent; and
c) identifying that the agent modulates an FSH or FSH Mimetic influenced cellular process or response when the activity of the product of the one or more FSH or FSH Mimetic stimulated genes in the cell sample in the presence of the agent differs from the activity of the product of the one or more FSH or FSH Mimetic stimulated genes in the absence of the agent.

3. A method for identifying an agent which modulates an FSH or FSH Mimetic influenced cellular process or response, the method comprising:
a) providing a sample of cells;
b) determining the level of expression in the sample of cells of one or more FSH or FSH Mimetic stimulated genes (Tables 1, 2, 3) in the presence and absence of a selected agent; and
c) identifying that the agent modulates an FSH or FSH Mimetic influenced cellular process or response when the expression of the one or more FSH or FSH Mimetic stimulated genes in the cell sample in the presence of the agent differs from the expression of the one or more FSH or FSH Mimetic stimulated genes in the absence of the agent.

4. A method for identifying an agent which modulates an FSH or FSH Mimetic influenced cellular process or response, the method comprising:
a) providing a sample of cells;
b) determining the activity in the sample of cells of the product of one or more FSH or FSH Mimetic stimulated genes (Table 1, 2, or 3) in the presence and absence of a selected agent; and
c) identifying that the agent modulates an FSH or FSH Mimetic influenced cellular process or response when the activity of the product of the one or more FSH or FSH Mimetic stimulated genes in the cell sample in the presence of the agent differs from the activity of the product of the one or more FSH or FSH Mimetic stimulated genes in the absence of the agent.

5. A method for detecting or monitoring a cellular process or response that is influenced by FSH or FSH Mimetic, the method comprising:
a) obtaining a sample of cells from a patient;
b) determining the level of expression in the sample of cells of one or more FSH or FSH Mimetic stimulated genes (Tables 1, 2, 3); and
c) identifying that the cells in the sample of cells obtained from the patient are undergoing a cellular process or response that is influenced by FSH or FSH Mimetic when the level of expression of the one or more FSH or FSH Mimetic stimulated genes in the cell sample is increased relative to the level of expression of the one or more FSH or FSH Mimetic stimulated genes in a control the sample.

6. A method for detecting or monitoring a cellular process or response that is influenced by FSH or FSH Mimetic, the method comprising:
a) obtaining a sample of cells from a patient;
b) determining the level of activity in the sample of cells of the product of one or more FSH or FSH Mimetic stimulated genes (Tables 1, 2, 3);
c) identifying that the cells in the sample of cells obtained from the patient are undergoing a cellular process or response that is influenced by FSH or FSH Mimetic when the level of activity of the product of the one or more FSH or FSH Mimetic stimulated genes in the cell sample is increased relative to the activity of the product of the one or more FSH or FSH Mimetic stimulated genes in a control the sample.

7. A method for assessing whether cells will be responsive to an agent which modulates an FSH or FSH Mimetic influenced cellular process or response comprising the steps of
a) exposing a sample of cells obtained from a patient to a test agent;
b) determining the level of expression in the sample of cells of the one or more FSH or FSH Mimetic stimulated genes (Tables 1, 2, and 3) in the sample exposed to the agent and in a sample of cells that is not exposed to the agent; and
c) determining that the cells will be responsive to the agent when 20 expression of the one or more of the FSH or FSH Mimetic stimulated genes is altered in the presence of the agent.

8. A method for assessing whether cells will be responsive to an agent which modulates an FSH or FSH Mimetic influenced cellular process or response comprising the steps of
a) exposing a sample of cells obtained from a patient to a test agent;
b) determining the level of activity of the product of the one or more FSH or FSH Mimetic stimulated genes (Tables 1, 2, and 3) in the sample of cells exposed to the agent and in a sample of cells that is not exposed to the agent; and
c) determining that the cells will be responsive to the agent when activity of the product of the one or more FSH or FSH Mimetic stimulated genes in the cell sample in the presence of the agent differs from the activity of the product of the one or more FSH or FSH Mimetic stimulated genes in the absence of the agent.

9. A method for modulating an FSH or FSH Mimetic influenced cellular process or response, the method comprising administering a compound which alters the expression or activity of an FSH or FSH Mimetic stimulated gene (Tables 1, 2, 3).

10. The method of claim 10 wherein said compound is a FSH or FSH Mimetic stimulated gene or the product thereof.

11. A method of treating a reproductive disorder or disease in a mammal, comprising the administration of a therapeutically effective dose of FSH or an FSH mimetic which alters the expression or activity of an FSH or FSH mimetic stimulated gene (Tables 1, 2, 3).
